(19) United States
(12) Patent Application Publication
(10) Pub. No.: US 2021/0087233 A1
(43) Pub. Date: Mar. 25, 2021

(54) DEUTERATED FORMS OF CARBETOCIN AND ANALOGS THEREOF

(71) Applicants: Christopher Bryant, Burr Ridge, IL (US); Joseph William Cormier, Bethesda, MD (US); Daniel G. Welch, Denver, CO (US)

(72) Inventors: Christopher Bryant, Burr Ridge, IL (US); Joseph William Cormier, Bethesda, MD (US); Daniel G. Welch, Denver, CO (US)

(21) Appl. No.: 16/952,251

(22) Filed: Nov. 19, 2020

(51) Int. Cl. C07K 7/56; C07B 59/00; A61K 9/00

(52) U.S. Cl. CPC C07K 7/56; A61K 38/00; A61K 9/0043; C07B 59/008

(57) ABSTRACT

The present disclosure relates to deuterated forms of oxytocin, carbetocin and analogs thereof. The disclosed deuterated compounds may have similar biochemical potency and selectivity as the parent compound, and, in select instances, the selective deuteration may enable substantial benefits to their overall therapeutic profile, such as reduced adverse side effects with a decreased metabolic liability, extended pharmacological effective life, enhanced subject compliance, and/or may also decrease population pharmacokinetic variability. The present disclosure also includes pharmaceutical compositions of deuterated compounds, which may be used to treat various diseases and conditions.

### FIELD OF THE DISCLOSURE

The present disclosure relates to deuterated forms of oxytocin, carbetocin, and analogs thereof. This disclosure also provides compositions comprising a deuterated compound of this disclosure. The disclosed compositions may be used in methods of treating diseases and conditions that are beneficially treated by administering oxytocin, carbetocin, and analogs thereof.

### BACKGROUND OF THE DISCLOSURE

Peptides, unlike proteins, have a shorter amino acid sequence, such as, for example, less than 50 amino acids. Because of this difference in size, peptides and proteins often possess different three-dimensional structures, properties, and functions. Peptides are used to treat various diseases and conditions, but their use is limited due to various factors. For example, peptides are known to be chemically and physically unstable and prone to hydrolysis and oxidation. Additionally, peptides generally have a tendency for aggregation, low membrane permeability, and a short half-life, and fast elimination. Also, peptides are not usually orally available. These aspects must be addressed for their use as medicines. (Fosgerau et al. (2015), Drug Discovery Today, January; 20(1):122-8.) Depending on potency, it may be necessary to formulate a peptide at a high concentration, but doing so may increase the likelihood of peptide aggregation. (Shire S. J. et al. (2004) J Pharm Sci. 93:1390-1402; Payne R. W. et al. (2006) Biopolymers 84:527-533.)

Oxytocin is an example of a peptide that demonstrates poor

chemical and physical stability, and has a short circulating plasma half-life. For example, oxytocin is known to degrade via a number of pathways, such as hydrolysis, isomerization, deamidation, oxidation, and dimerization. (Zhao, L. et al. (2001) Int. J. Pharm. 218, 43-56.) The amino acids and/or amino acid sites more likely to undergo degradation pathways in oxytocin are illustrated below.

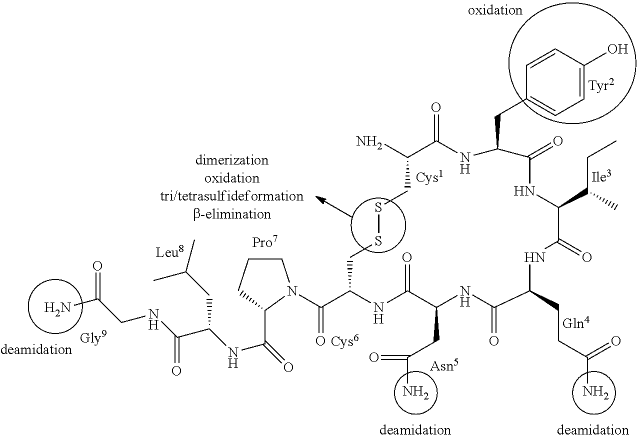 [SMILES: CC[C@H](C)[C@@H]1NC(=O)[C@H](Cc2ccc(O)cc2)NC(=O)[C@@H](N)CSSC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

Carbetocin [(1-desamino-1-monocarba-2(O-methyl)-tyrosine) oxytocin] is a long-acting synthetic oxytocin analog. Carbetocin is an uncharged peptide. The structure of carbetocin is shown below.

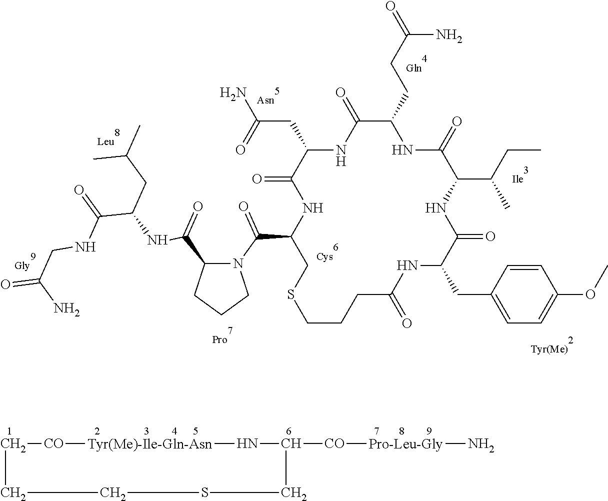

Carbetocin is an unusual peptide: it is small (8 amino acids), possesses no charge, is cyclic, and is highly lipophilic. It is known that carbetocin lacks a stable and well-defined tertiary structure. Carbetocin is currently used outside the U.S. to treat or prevent postpartum hemorrhage during or following caesarean section. As such, carbetocin is administered by slow intravenous (IV) single injection at a dose of 100 μg. This formulation (Duratocin®, Ferring) requires refrigeration and contains 0.1 mg/mL of carbetocin, 9 mg sodium chloride, acetic acid-glacial to pH 3.8 and water for injection to 1 mL. (Widmer M. et al. (2016) Trials. 17:143.) Carbetocin (IV form) is currently registered in more than 70 countries under the trade names PABAL/DURATOCIN/LONACTENE/DURATOBAL.

Given potential absorption, distribution, metabolism and/or excretion (ADME) considerations associated with peptides, there remains a need to provide improved compounds that have the beneficial activities of oxytocin and carbetocin, and may also have other benefits, e.g., reduced adverse side effects, with a decreased metabolic liability, to further extend their pharmacological effective life, enhance subject compliance, and, potentially, to decrease population pharmacokinetic variability and/or decrease their potential for dangerous drug-drug interactions.

According to the present disclosure, selective incorporation of deuterium in place of hydrogen (deuteration) may have the beneficial effect of retaining the biochemical potency and selectivity of physiologically active compounds, such as peptides, while, in select instances, may further enable substantial benefits to their overall therapeutic profile (e.g., positively impacting their metabolic rate, safety, efficacy, tolerability of a therapeutic agent). A longer duration of a drug (e.g. peptide) in the body may provide continued beneficial effects.

### SUMMARY OF THE DISCLOSURE

The present disclosure is directed to novel deuterated compounds which can have, in some instances, a better overall therapeutic profile than the parent compound (i.e. an undeuterated compound). For example, the disclosed deuterated compounds may show reduced adverse side effects, with a decreased metabolic liability, and/or enhanced subject compliance. In some embodiments, the selective deuteration of the parent compound may decrease population pharmacokinetic variability.

In at least some embodiments, the present disclosure is directed to a deuterated compound of formula (A), or a pharmaceutically acceptable salt, stereoisomer, solvate, or hydrate thereof:

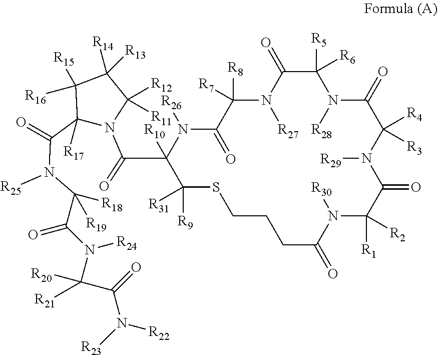

wherein:
R₁, R₃, R₅, R₇, R₉, R₁₀, R₁₁, R₁₂, R₁₃, R₁₄, R₁₅, R₁₆, R₁₇, R₁₈, R₂₀, R₂₁, R₂₂, R₂₃, R₂₄, R₂₅, R₂₆, R₂₇, R₂₈, R₂₉, R₃₀, and R₃₁ are independently chosen from H and D;
R₂ is chosen from

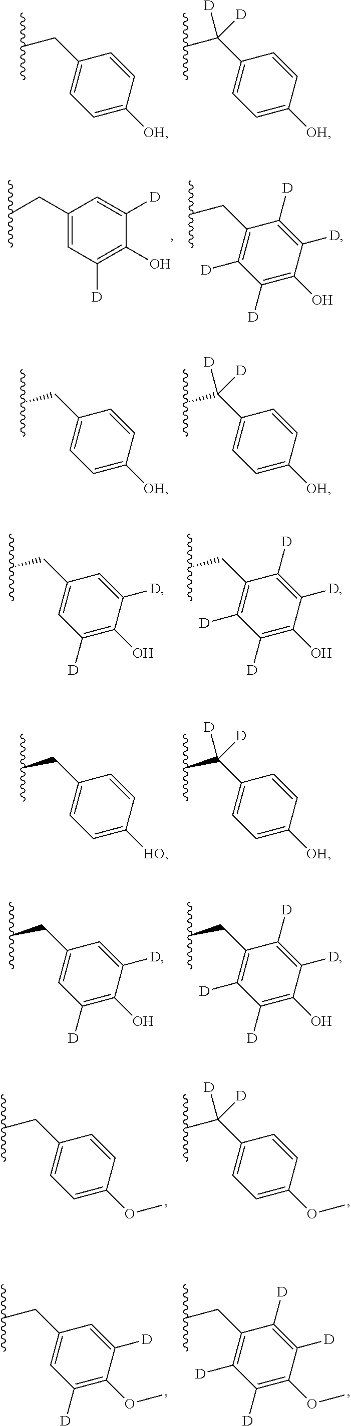

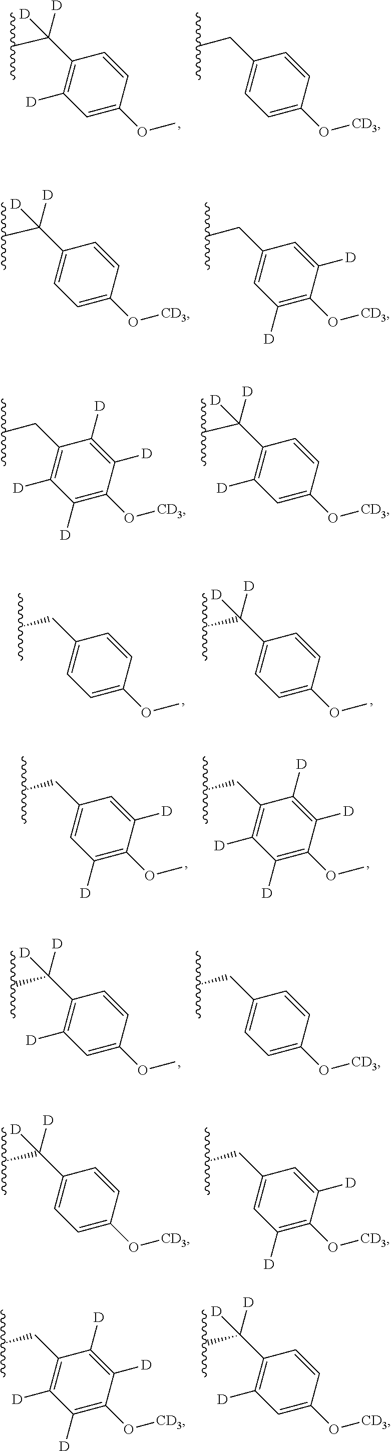

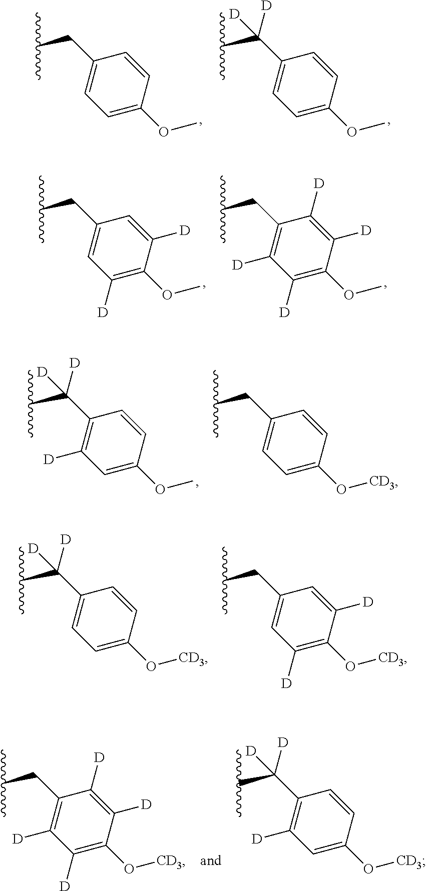

R₄ is chosen from

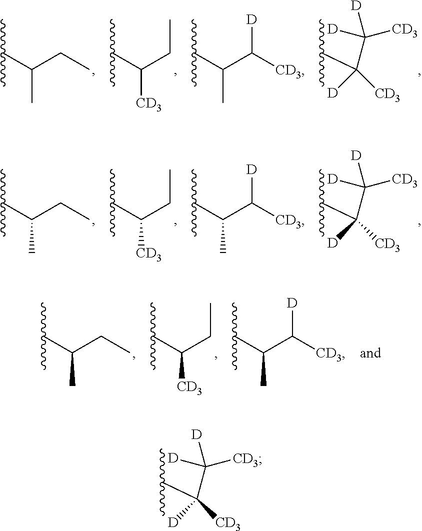

R₆ is chosen from

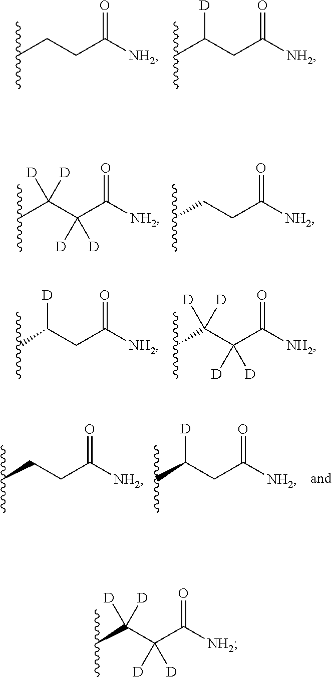

R₈ is chosen from

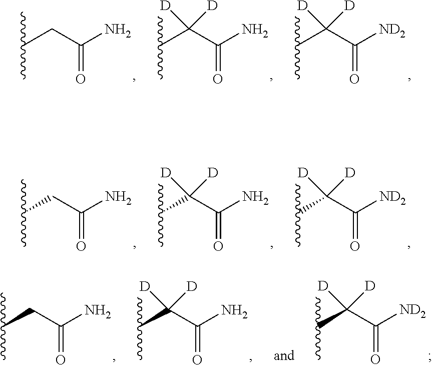

and
R₁₉ is chosen from

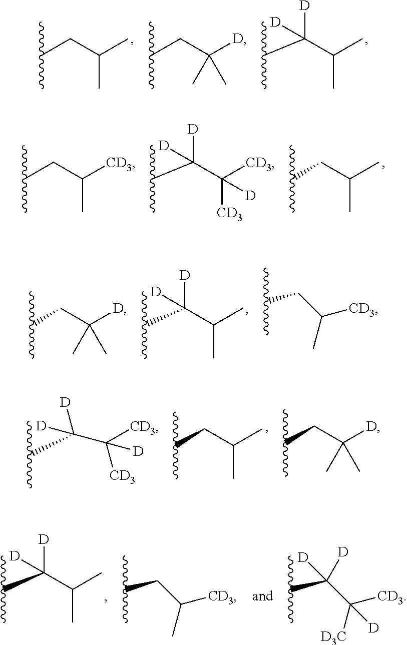

In at least one embodiment, the compound of Formula (A) is a compound of Formula (Aa), or a pharmaceutically acceptable salt, stereoisomer, solvate, or hydrate thereof:

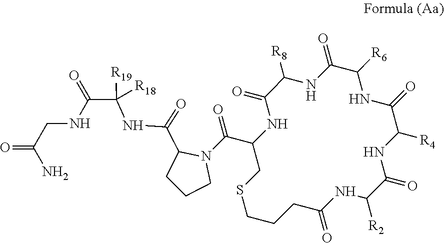

wherein:
R₁₈ is chosen from H and D;
R₂ is chosen from

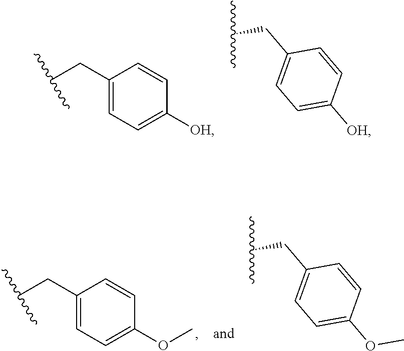

R₄ is chosen from

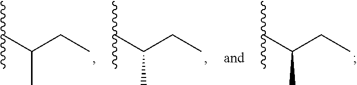

R₆ is chosen from

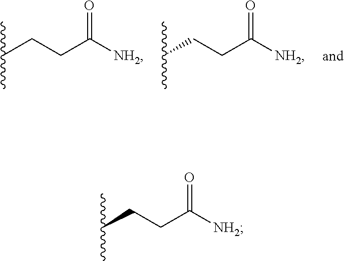

R₈ is chosen from

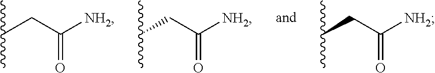

R₁₉ is chosen from

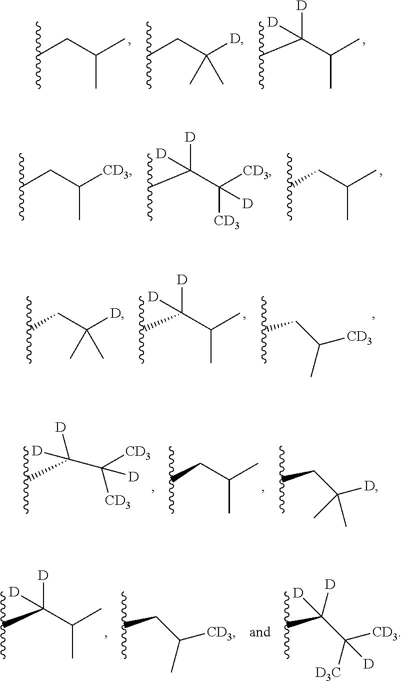

In some embodiments, the deuterated compound of Formula (A) or (Aa) is chosen from:

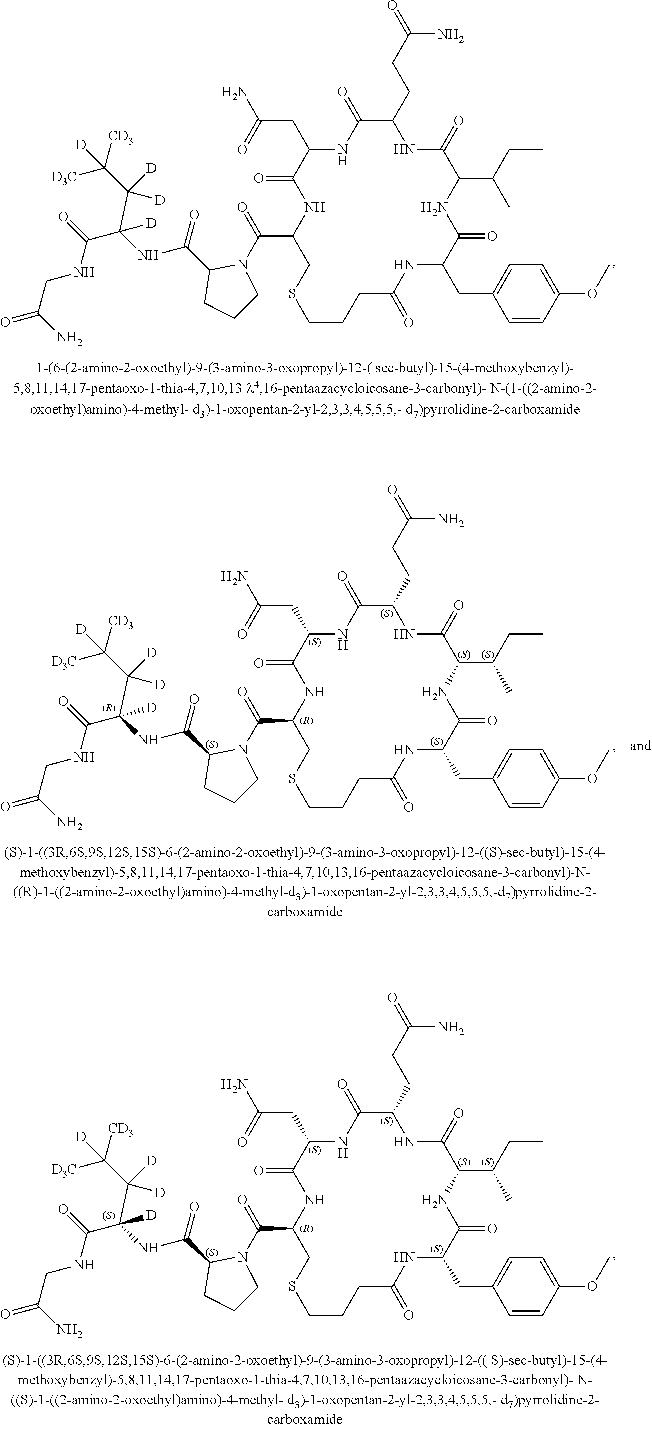

or a pharmaceutically acceptable salt, stereoisomer, solvate, or hydrate thereof.

In at least one embodiment, the deuterated compound of Formula (A) or Formula (Aa) is (S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-(methyl-d3)-1-oxopentan-2-yl-2,3,3,4,5,5,5-d7)pyrrolidine-2-carboxamide (i.e. Carbetocin-d₁₀ or [Leu-d₁₀]-Carbetocin).

In at least some embodiments, the deuterated compound of Formula (A) or Formula (Aa) may be used in a pharmaceutical composition of the present disclosure. In some embodiments, the pharmaceutical composition may be administered intranasally on a daily basis for a period of time, such as at least 3 weeks. In at least one embodiment, the pharmaceutical composition can be administered intranasally up to 3 times daily for chronic use.

In at least one embodiment, the pharmaceutical composition of the present disclosure may be for use in (or in the manufacture of medicaments for) the treatment or prevention of a neurodevelopmental disorder or related symptoms in a subject in need thereof. In at least one embodiment, a therapeutically-effective amount of a pharmaceutical composition of the present disclosure may be administered to a subject diagnosed with Prader-Willi syndrome. In one embodiment, the pharmaceutical composition may be administered to the subject intranasally. In at least one embodiment, a total daily dose of the deuterated compound of Formula (A) may be from about 1 mg/day to about 30 mg/day. In at least one embodiment, a total daily dose of the deuterated compound of Formula (A) may be from about 8.0 mg/day to about 30.0 mg/day. In at least one embodiment, a total daily dose of the deuterated compound of Formula (A) may be from about 9.0 mg/day to about 29.0 mg/day. In one embodiment, a total daily dose of the deuterated compound of Formula (A) may be chosen from about 8.0 mg/day, about 9.0 mg/day, 10.0 mg/day, about 11.0 mg/day, about 12.0 mg/day, about 13.0 mg/day, about 14.0 mg/day, 15.0 mg/day. 16.0 mg/day, 17.0 mg/day, 18.0 mg/day, 19.0 mg/day, 20.0 mg/day, 21.0 mg/day, 22.0 mg/day, 23.0 mg/day, 24.0 mg/day, 25.0 mg/day, 26.0 mg/day, 27.0 mg/day, 28.0 mg/day, 29.0 mg/day, and about 30.0 mg/day. In another embodiment, a total daily dose of the deuterated compound of Formula (A) may be chosen from about 9.1 mg/day, about 9.2 mg/day, about 9.3 mg/day, about 9.4 mg/day, about 9.5 mg/day, about 9.6 mg/day, about 9.7 mg/day, about 9.8 mg/day, and about 9.9 mg/day. In at least one embodiment, a total daily dose of the deuterated compound of Formula (A) can be 9.6 mg/day. In one embodiment, a total daily dose of the deuterated compound of Formula (A) may be chosen from about 11.1 mg/day, about 11.2 mg/day, about 11.3 mg/day, about 11.4 mg/day, about 11.5 mg/day, about 11.6 mg/day, about 11.7 mg/day, about 11.8 mg/day, and about 11.9 mg/day. In at least one embodiment, a total daily dose of the deuterated compound of Formula (A) may be 11.4 mg/day. In one embodiment, a total daily dose of the deuterated compound of Formula (A) may be chosen from about 28.1 mg/day, about 28.2 mg/day, about 28.3 mg/day, about 28.4 mg/day, about 28.5 mg/day, about 28.6 mg/day, about 28.7 mg/day, about 28.8 mg/day, and about 28.9 mg/day. In at least one embodiment, a total daily dose of the deuterated compound of Formula (A) can be 28.8 mg/day. In at least one embodiment, the total daily dose may be divided into 3 equal doses. In some embodiments, the pharmaceutical compositions comprising a deuterated compound of Formula (A) may lead to reduced adverse side effects, decreased metabolic liability, enhanced subject compliance, and/or decreased population pharmacokinetic variability.

### BRIEF DESCRIPTION OF DRAWINGS

The foregoing summary, as well as the following detailed description of the disclosure, will be better understood when read in conjunction with the appended drawings. For the purpose of illustrating the present disclosure, the attached drawings illustrate some, but not all, alternative embodiments. It should be understood, however, that the disclosure is not limited to the precise arrangements and instrumentalities shown. These figures, which are incorporated into and constitute part of the specification, assist in explaining the principles of the disclosure.

FIG. 1. shows a chromatogram of [Leu-d₁₀]-Carbetocin described in the Example.

FIG. 2. shows an expansion of the major peaks present in the chromatogram of [Leu-d₁₀]-Carbetocin. Peak 3 is [Leu-d₁₀]-Carbetocin.

### DETAILED DESCRIPTION OF THE DISCLOSURE

The present disclosure relates to deuterated forms of oxytocin, carbetocin, and analogs thereof. This disclosure also provides compositions comprising a deuterated compound of this disclosure. The disclosed compositions may be used in methods of treating diseases and conditions that are beneficially treated by administering oxytocin, carbetocin, and analogs thereof. The disclosed deuterated compounds of Formula (A) and Formula (Aa) may be more resistant to degradation pathways (e.g. hydrolysis, isomerization, deamidation, oxidation, and dimerization) associated with their parent compounds. The disclosed novel compounds are deuterated forms of carbetocin, or analogs thereof, and may have a better overall therapeutic profile than the parent compounds. For example, in select instances, the disclosed deuterated compounds may increase potency of the parent compound (e.g. carbetocin, oxytocin) and/or may also overcome the potential disadvantages associated with peptides. In some embodiments, the disclosed deuterated compounds of Formula (A) and Formula (Aa) may show a reduction in adverse side effects, a decrease in metabolic liability, an enhanced subject compliance, and/or, potentially, a decreased population pharmacokinetic variability.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.).

Isomers

Compounds containing one or more chiral centers may be used in enantiomerically or diastereoisomerically pure form, in the form of racemic mixtures and in the form of mixtures enriched in one or more stereoisomers. The compounds of the present disclosure encompass the racemic form of the compounds as well as the individual enantiomers, diastereomers, and stereoisomerically-enriched mixtures.

Conventional techniques for the preparation and/or isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of Formula (A) and Formula (Aa), (and chiral precursors thereof) may be obtained in enantiomerically-enriched form using chromatography, typically HPLC.

The compounds of Formula (A) and Formula (Aa) may exhibit the phenomena of tautomerism and structural isomerism. Tautomers exist as mixtures of a tautomeric set in solution. In solid form, usually one tautomer predominates. Even though only one tautomer may be depicted herein, the present disclosure encompasses all possible tautomers of the compounds of Formula (A) and Formula (Aa).

The compounds of the present disclosure may contain an asymmetric carbon atom, for example, as the result of deuterium substitution or otherwise. As such, compounds of this disclosure can exist as either individual enantiomers, or mixtures of the two enantiomers. Accordingly, a compound of the present disclosure will include both racemic mixtures, and also individual respective stereoisomers that are substantially free from another possible stereoisomer.

Isotopes

It will be recognized that some variation of natural isotopic abundance occurs in a synthesized compound depending upon the origin of chemical materials used in the synthesis.

As used herein, the term deuterated compounds embrace compounds where in a particular position at least one hydrogen atom is replaced by deuterium (D or ²H). In a compound of this disclosure, when a particular position is designated as having deuterium, it is understood that the abundance of deuterium at that position is substantially greater than the natural abundance of deuterium, which is approximately 0.015%. In one embodiment, a position designated as having deuterium has a minimum isotopic enrichment factor of at least 3000 (45% deuterium incorporation) at each atom designated as deuterium in said compound.

As used herein, the term “isotopic enrichment factor” means the ratio between the isotopic abundance and the natural abundance of a specified isotope. In other embodiments, a compound of this disclosure has an isotopic enrichment factor for each designated deuterium atom of, but not limited to, at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this disclosure, any atom not specifically designated as a particular isotope is meant to represent any stable isotope of that atom. Unless otherwise stated, when a position is designated specifically as “H” or “hydrogen,” the position is understood to have hydrogen present at its natural isotopic abundance.

Isotopically-labeled compounds of the present disclosure can generally be prepared by conventional techniques known to those skilled in the art or by processes analogous to those described herein, using an appropriate isotopically-labeled reagent in place of the non-isotopically labeled reagent otherwise employed.

The term “compound,” as used herein, is also intended to include any salts, solvates, or hydrates thereof.

As used herein, a salt of a compound of this disclosure may be formed between an acid and a basic group of the compound, such as an amino functional group, or a base and an acidic group of the compound, such as a carboxyl functional group. According to another embodiment, the compound is a pharmaceutically acceptable acid addition salt.

As used herein, the term “pharmaceutically acceptable” refers to a component that is, within the scope of sound medical judgment, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to a recipient, is capable of providing, either directly or indirectly, a compound of this disclosure. A “pharmaceutically acceptable counterion” is an ionic portion of a salt that is not toxic when released from the salt upon administration to a recipient.

In some embodiments, the disclosed compounds of Formula (A) and Formula (Aa) may be obtained as salts. In at least some embodiments, non-limiting examples of acids that may be employed to form pharmaceutically acceptable salts include inorganic and organic acid addition salts. In some embodiments, a suitable acid addition salt is formed from an acid which forms a non-toxic salt, for example, hydrochloride, hydrobromide, hydroiodide, sulphate, hydrogen sulphate, nitrate, phosphate, and hydrogen phosphate salts. In other embodiments, additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. In other embodiments, suitable base salts are formed from bases that form non-toxic salts, for example, aluminum, calcium, lithium, magnesium, potassium, sodium, zinc and diethanolamine salts. In some embodiments, the compounds of Formula (A) and (Aa) are organic acid salts chosen from acetate and trifluoroacetate. In one embodiment, the compounds of Formula (A) and (Aa) are trifluoroacetate salts.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

As used herein, “D” refers to deuterium.

Compounds of Formula (A)

In at least some embodiments, the present disclosure is directed to a deuterated compound of formula (A), or a pharmaceutically acceptable salt, stereoisomer, solvate, or hydrate thereof:

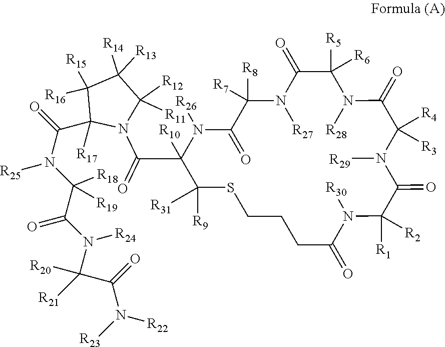

wherein:
R₁, R₃, R₅, R₇, R₉, R₁₀, R₁₁, R₁₂, R₁₃, R₁₄, R₁₅, R₁₆, R₁₇, R₁₈, R₂₀, R₂₁, R₂₂, R₂₃, R₂₄, R₂₅, R₂₆, R₂₇, R₂₈, R₂₉, R₃₀, and R₃₁ are independently chosen from H, F, and D;
R₂ is chosen from

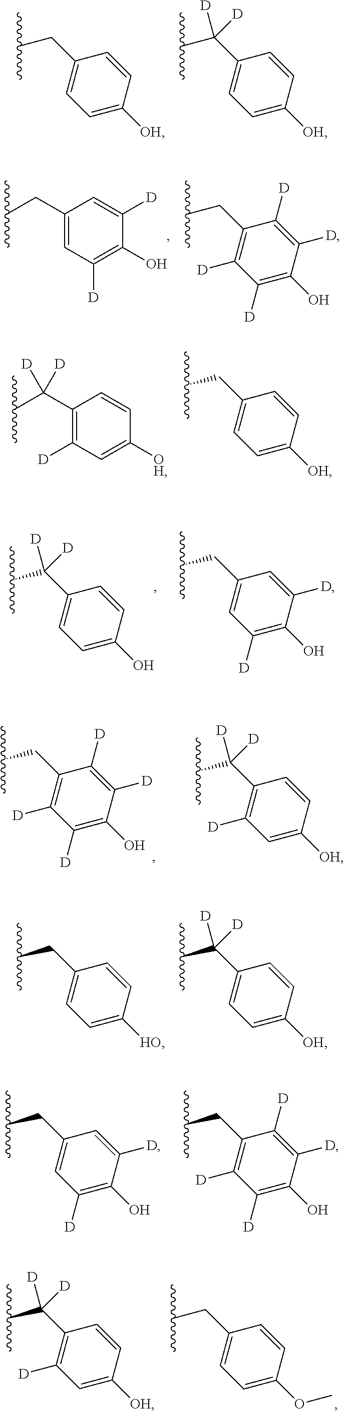

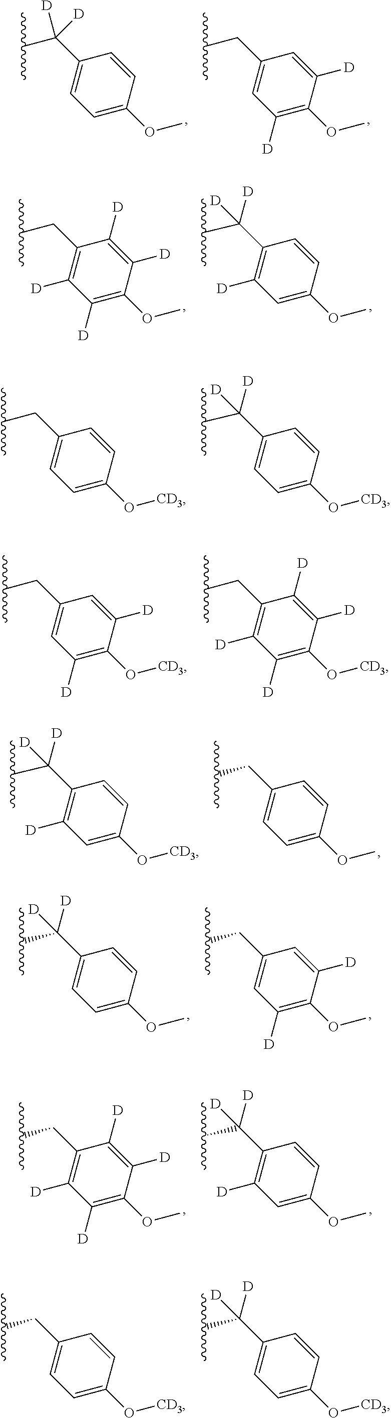

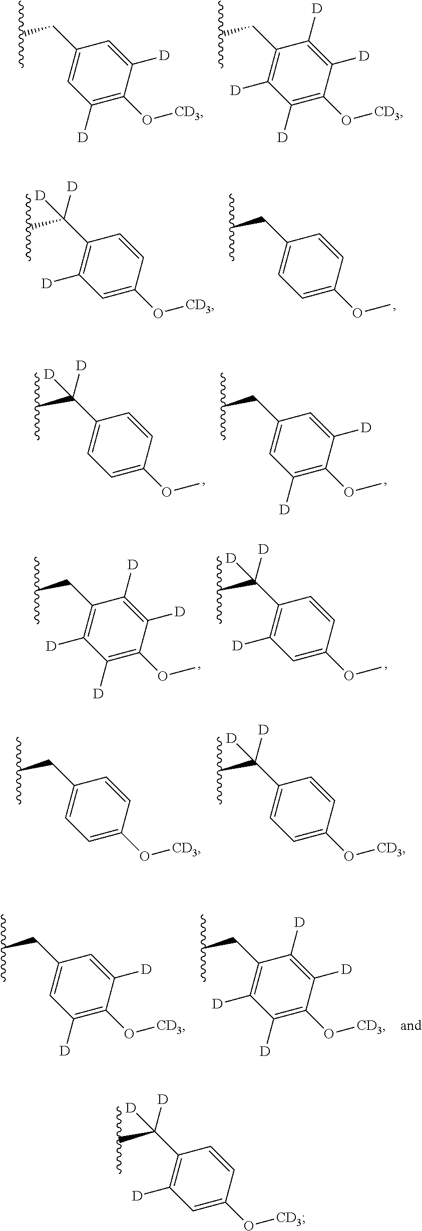

R₄ is chosen from

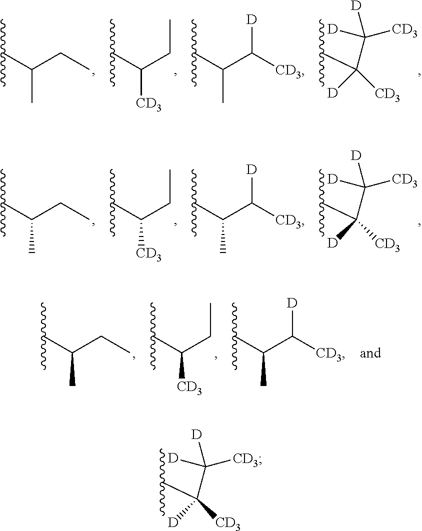

R₆ is chosen from

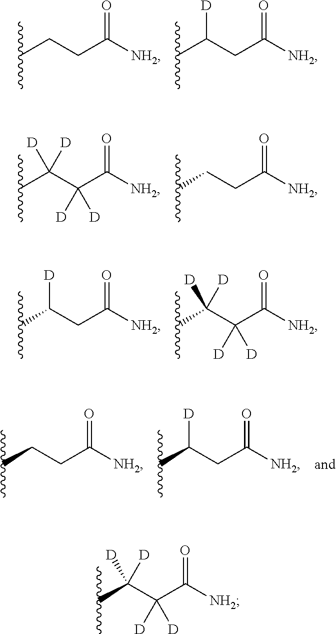

R₈ is chosen from

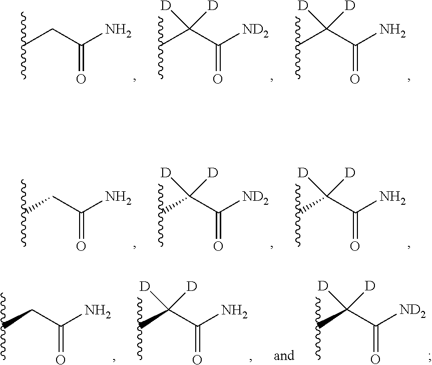

and
R₁₉ is chosen from

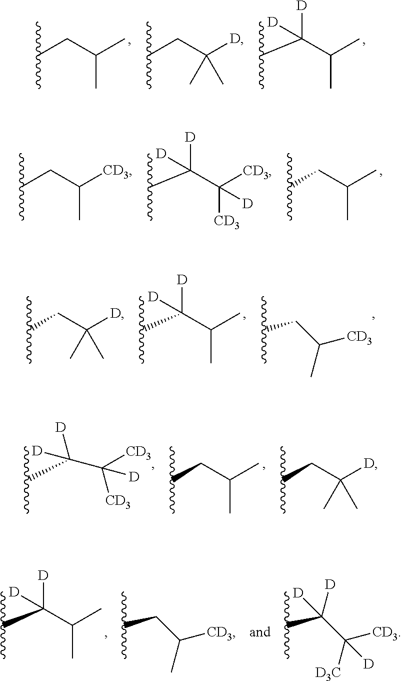

In some embodiments, R₁, R₃, R₅, R₇, R₉, R₁₀, R₁₁, R₁₂, R₁₃, R₁₄, R₁₅, R₁₆, R₁₇, R₁₈, R₂₀, R₂₁, R₂₂, R₂₃, R₂₄, R₂₅, R₂₆, R₂₇, R₂₈, R₂₉, R₃₀, and R₃₁ may be independently chosen from H, F, and D. In one embodiment, R₁, R₃, R₅, R₇, R₉, R₁₀, R₁₁, R₁₂, R₁₃, R₁₄, R₁₅, R₁₆, R₁₇, R₁₈, R₂₀, R₂₁, R₂₂, R₂₃, R₂₄, R₂₅, R₂₆, R₂₇, R₂₈, R₂₉, R₃₀, and R₃₁ may be independently chosen from H and D. In at least some embodiments, R₁₈ is D.

In some embodiments, R₂ is chosen from a side chain of a natural or unnatural amino acid. In some embodiments, the amino acid may be an amino acid with a hydrophobic side chain. In at least some embodiments, the amino acid is chosen from tyrosine and O-Methyltyrosine. In one embodiment, the amino acid is 4-methoxy-L-phenylalanine (i.e. O-Methyl-L-tyrosine). In other embodiments, the amino acid may be a N-α-Fmoc protected amino acid, such as N-α-Fluorenylmethoxycarbonyl-O-methyl-L-tyrosine (Fmoc-Tyr(Me)-OH). In other embodiments, the side chains of 4-methoxy-L-phenylalanine and Fmoc-Tyr(Me)-OH may be deuterated. In at least some embodiments, the deuterated tyrosine derivatives may be chosen from Tyrosine-3,3-d2 (linear formula is 4-(HO)C₆H4CD₂CH(NH₂)CO₂H), Tyrosine-(phenyl-3,5-d₂) (linear formula is 4-(HO)C₆H₂D₂CH₂CH(NH₂)CO₂H), Tyrosine-(phenyl-d₄) (linear formula is 4-(HO)C₆D₄CH₂CH(NH₂)CO₂H), and Tyrosine-α,β,β,2,3,5,6-d₇ (linear formula is 4-(HO)C₆D₄CD₂CD(NH₂)CO₂H).

In some embodiments, R₂ may be chosen from

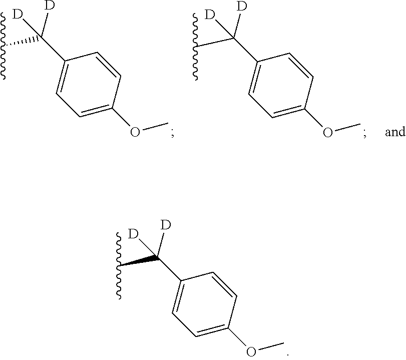

In one embodiment, R₂ is

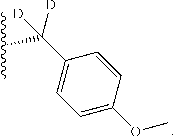

In one embodiment, R₄ is chosen from a side chain of a natural or unnatural amino acid. In some embodiments, the amino acid may be an amino acid with a hydrophobic side chain. In at least some embodiments, the amino acid is isoleucine.

In other embodiments, the amino acid may be a N-α-Fmoc protected amino acid, such as N-α-Fluorenylmethoxycarbonyl-L-isoleucine (Fmoc-Ile-OH). In other embodiments, the side chains of isoleucine and Fmoc-Ile-OH may be deuterated. In at least some embodiments, the deuterated isoleucine derivatives may be chosen from Isoleucine-2-d₁ (linear formula is CH₃CH₂CH(CH₃)CD(NH₂)COOH), Isoleucine-3-methyl-d₃ (linear formula is CH₃CH₂CH(CD₃)CD(NH₂)COOH), Isoleucine-4,5,5,5-d4 (linear formula is CD₃CDHCH(CD₃)CD(NH₂)COOH), and Isoleucine-d₁₀ (linear formula is CD₃CD₂CD(CD₃)CD(NH₂)COOH).

In some embodiments, R₄ may be chosen from

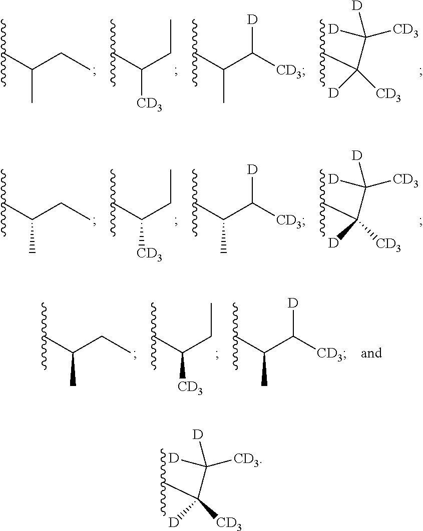

In some embodiments, R₄ may be chosen from

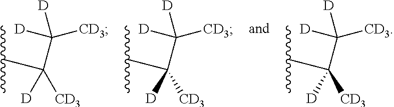 [SMILES: *C([2H])(C([2H])([2H])[2H])C([2H])([2H])C([2H])([2H])[2H] ; *[C@@]([2H])(C([2H])([2H])[2H])C([2H])([2H])C([2H])([2H])[2H] ; *[C@]([2H])(C([2H])([2H])[2H])C([2H])([2H])C([2H])([2H])[2H]]

In one embodiment, R₄ is

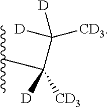 [SMILES: *[C@@]([2H])(C([2H])([2H])[2H])C([2H])([2H])C([2H])([2H])[2H]]

In one embodiment, R₆ is chosen from a side chain of a natural or unnatural amino acid. In some embodiments, the amino acid may be an amino acid with a polar uncharged side chain. In at least some embodiments, the amino acid is glutamine. In other embodiments, the amino acid may be a N-α-Fmoc protected amino acid, such as N-α-Fluorenylmethoxycarbonyl-γ-trityl-L-glutamine (Fmoc-Gln(Trt)-OH). In other embodiments, the side chains of glutamine and Fmoc-Gln(Trt)-OH may be deuterated. In at least some embodiments, the deuterated glutamine derivative may be Glutamine-2,3,3,4,4-d₅ (linear formula is H₂NCO(CD₂)₂CD(NH₂)CO₂H).

In some embodiments, R₆ may be chosen from

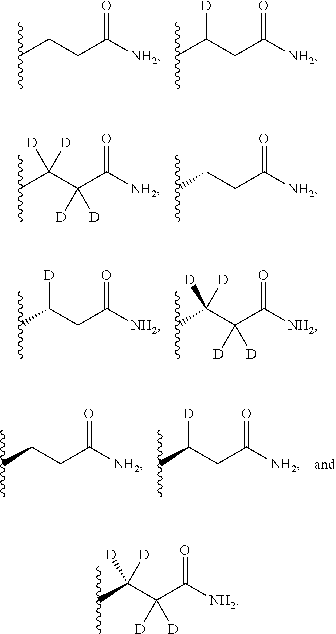

In some embodiments, R₆ may be chosen from

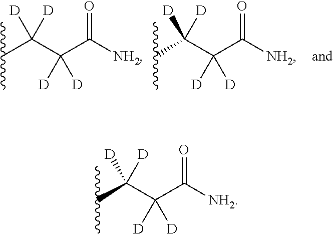

In one embodiment, R₆ is

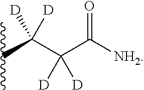

In at least some embodiments, R₈ is chosen from a side chain of a natural or unnatural amino acid. In some embodiments, the amino acid may be an amino acid with a polar uncharged side chain. In at least some embodiments, the amino acid is asparagine. In other embodiments, the amino acid may be a N-α-Fmoc protected amino acid, such as N-α-Fluorenylmethoxycarbonyl-N-γ-trityl-L-asparagine (Fmoc-Asn(Trt)-OH). In other embodiments, the side chains of asparagine and Fmoc-Asn(Trt)-OH may be deuterated. In at least some embodiments, the deuterated asparagine may be Asparagine-d₃.

In some embodiments, R₈ may be chosen from

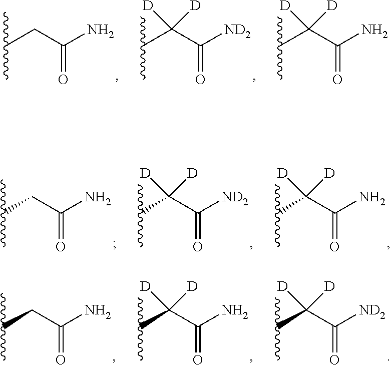

In some embodiments, R₈ may be chosen from

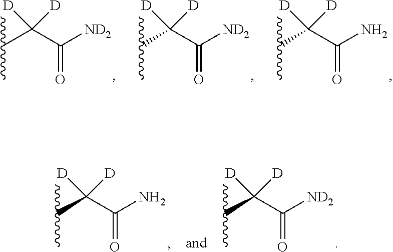

In one embodiment, R₈ is

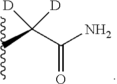

In some embodiments, R₁₉ is chosen from a side chain of a natural or unnatural amino acid. In one embodiment, the amino acid may be an amino acid with a hydrophobic side chain. In at least some embodiments, the amino acid is leucine. In other embodiments, the amino acid may be a N-α-Fmoc protected amino acid, such as N-α-Fluorenylmethoxycarbonyl-L-leucine (Fmoc-Leu-OH). In other embodiments, the side chains of leucine and Fmoc-Leu-OH may be deuterated. In at least some embodiments, the deuterated leucine derivatives may be chosen from Leucine-4-d₁ (linear formula is (CH₃)₂CDCH₂CH(NH₂)CO₂H), Leucine-3,3-d2 (linear formula is (CH₃)₂CHCD₂CH(NH₂)COOH), Leucine-5,5,5-d₃ (linear formula is CH₃CH(CD₃)CH₂CH(NH₂)CO₂H), N-(9-Fluorenylmethoxycarbonyl)-L-leucine-5,5,5-d₃ (Fmoc-Leu-OH-5,5,5-d₃), Leucine-2,3,3,4,5,5,5,5′,5′,5′-d₁₀ (linear formula is (CD₃)₂CDCD₂CD(NH₂)CO₂H), N-(9-Fluorenylmethoxycarbonyl)-L-leucine-d₁₀ (Fmoc-Leu-OH-d₁₀), and DL-Leucine-d₁₀-N-Fmoc.

In some embodiments, R₁₉ may be chosen from

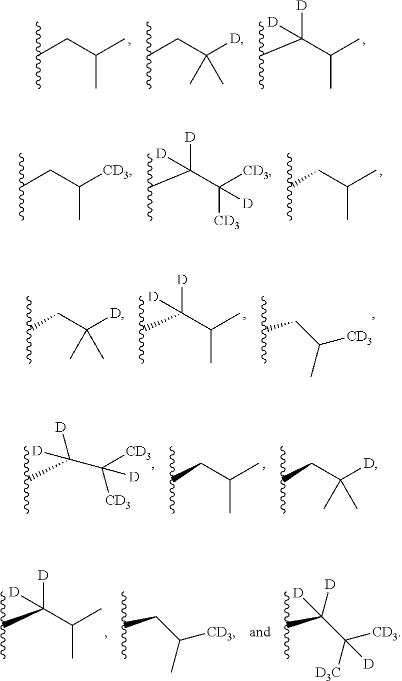

In some embodiments, R₁₉ may be chosen from

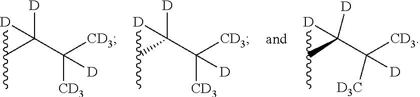

In one embodiment, R₁₉ is

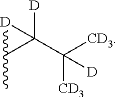 [SMILES: *C([2H])([2H])C([2H])(C([2H])([2H])[2H])C([2H])([2H])[2H]]

In at least some embodiments, oxytocin or an oxytocin analog, such as carbetocin or a carbetocin derivative of Formula (A), may comprise one or more deuterium atoms at any position of the compound of Formula (A) where the one or more deuterium atoms can be incorporated into the disclosed compounds.

In at least some embodiments, the present disclosure is directed to a deuterated compound of formula (Aa), or a pharmaceutically acceptable salt, stereoisomer, solvate, or hydrate thereof:

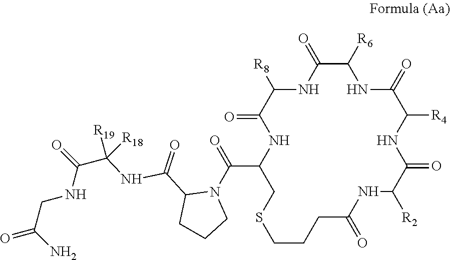

wherein:
R₁₈ is chosen from H, halogen, and D;
R₂ is chosen from

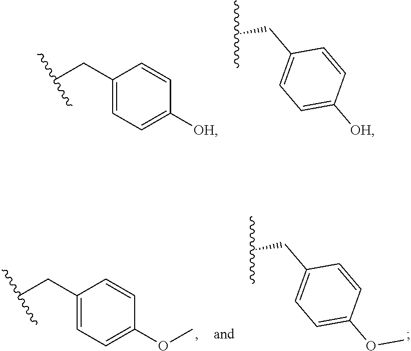

R₄ is chosen from

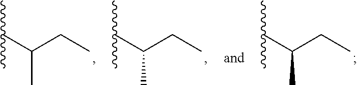

R₆ is chosen from

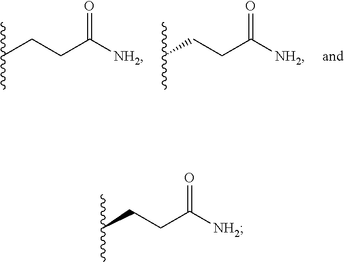

R₈ is chosen from

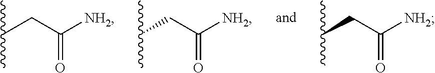

R₁₉ is chosen from

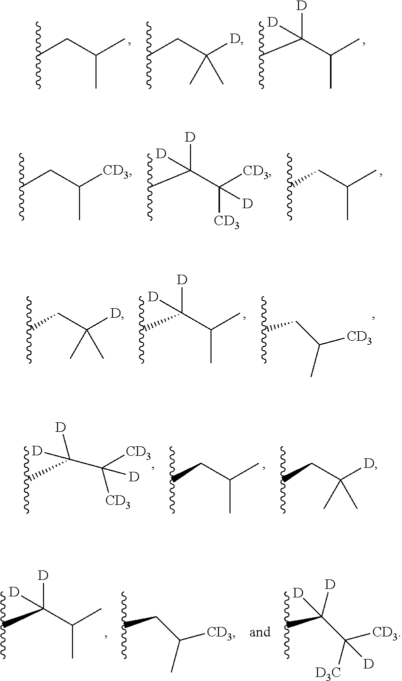

In one embodiment, R₁₈ from Formula (Aa) may be chosen from H, F, and D. In one embodiment, R₁₈ may be chosen from H and D. In one embodiment, R₁₈ is D.

In some embodiments, R₁₉ may be chosen from

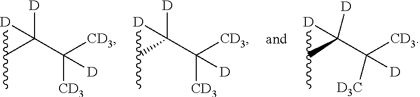

In one embodiment, R₁₉ is

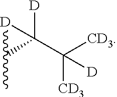

In another embodiment, R₂ is chosen from

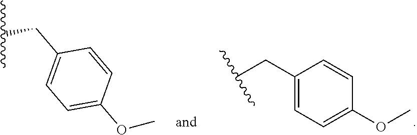

In one embodiment, R₄ is chosen from

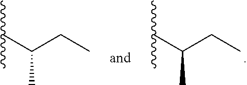

In one embodiment, R₄ is

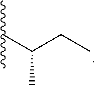 [SMILES: *[C@@H](C)CC]

In one embodiment, R₆ is chosen from

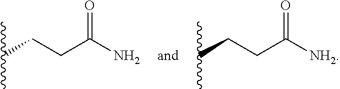

In one embodiment, R₆ is

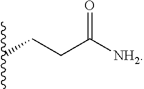

In one embodiment, R₈ is chosen from

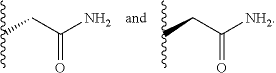

In one embodiment, R₈ is

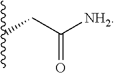

In at least some embodiments, a carbetocin derivative of Formula (Aa), may comprise one or more deuterium atoms at any position of the compound of Formula (Aa) where the one or more deuterium atoms can be incorporated into the disclosed compounds.

In at least some embodiments, a deuterated compound of formula (A) is chosen from:

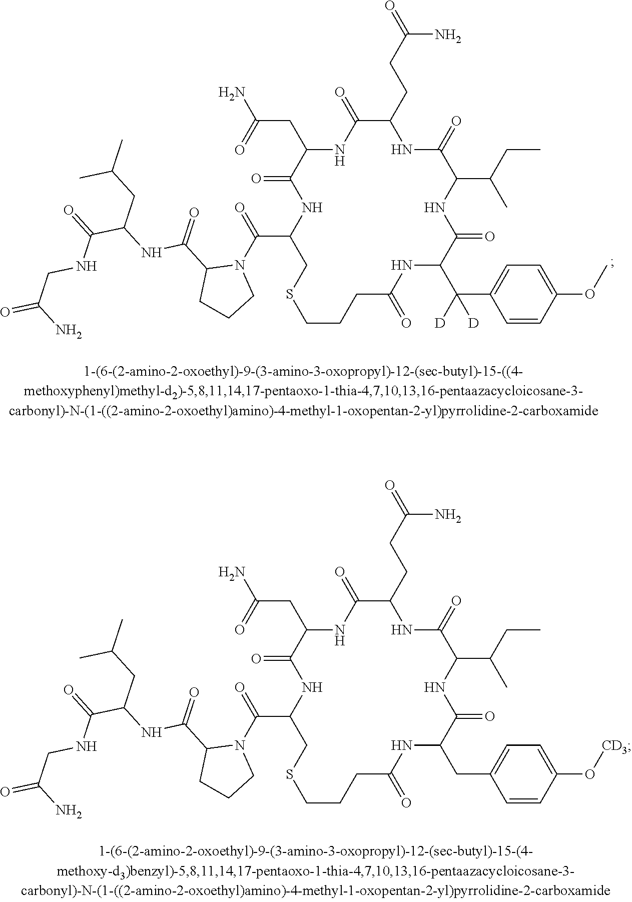 [SMILES: [2H]C([2H])(c1ccc(OC)cc1)C1NC(=O)CCCSCC(C(=O)N2CCCC2C(=O)NC(CC(C)C)C(=O)NCC(N)=O)NC(=O)C(CC(N)=O)NC(=O)C(CCC(N)=O)NC(=O)C(C(C)CC)NC1=O ; [2H]C([2H])([2H])Oc1ccc(CC2NC(=O)CCCSCC(C(=O)N3CCCC3C(=O)NC(CC(C)C)C(=O)NCC(N)=O)NC(=O)C(CC(N)=O)NC(=O)C(CCC(N)=O)NC(=O)C(C(C)CC)NC2=O)cc1]

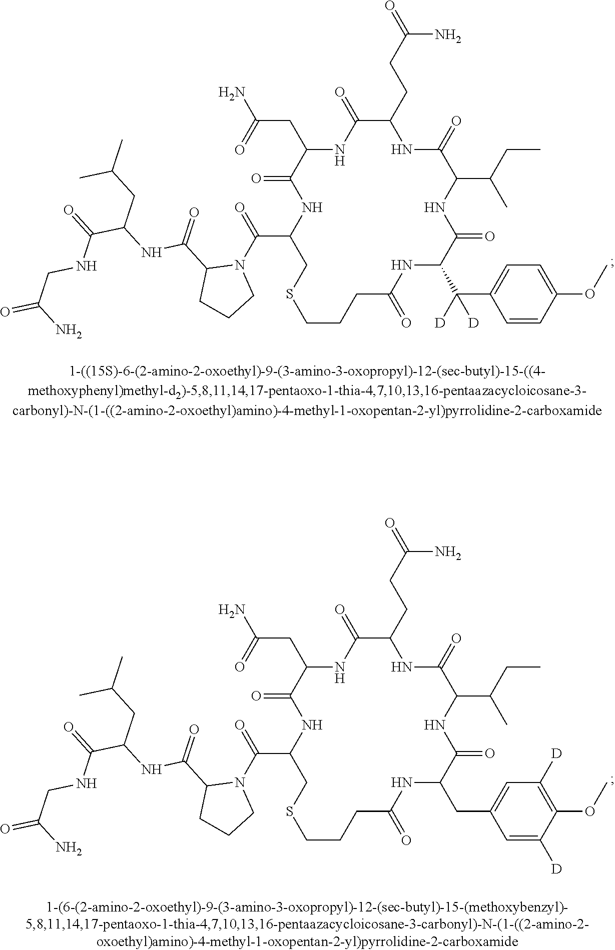 [SMILES: [2H]C([2H])(c1ccc(OC)cc1)[C@@H]1NC(=O)CCCSCC(C(=O)N2CCCC2C(=O)NC(CC(C)C)C(=O)NCC(N)=O)NC(=O)C(CC(N)=O)NC(=O)C(CCC(N)=O)NC(=O)C(C(C)CC)NC1=O ; [2H]c1cc(CC2NC(=O)CCCSCC(C(=O)N3CCCC3C(=O)NC(CC(C)C)C(=O)NCC(N)=O)NC(=O)C(CC(N)=O)NC(=O)C(CCC(N)=O)NC(=O)C(C(C)CC)NC2=O)cc([2H])c1OC]

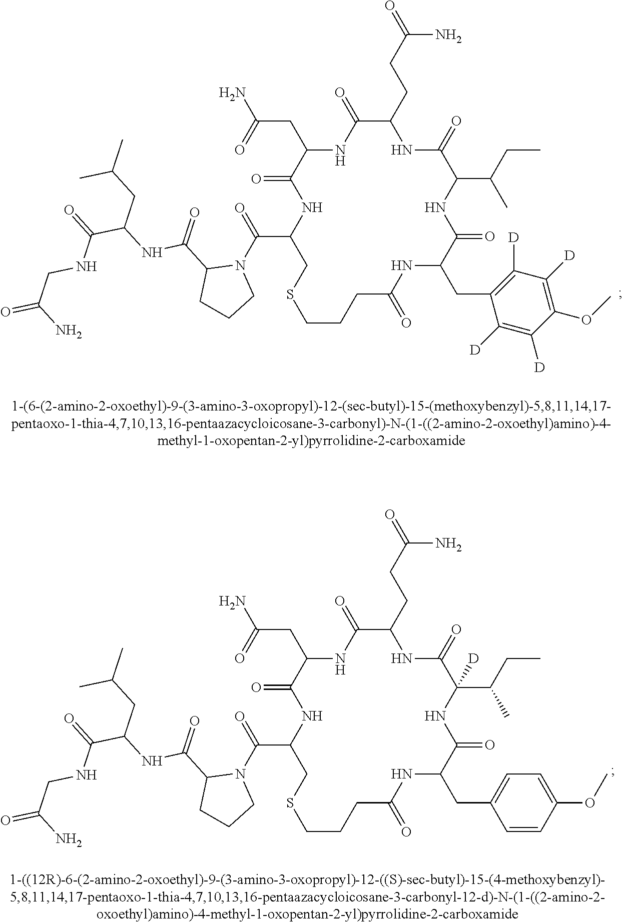 [SMILES: [2H]c1c([2H])c(OC)c([2H])c([2H])c1CC1NC(=O)CCCSCC(C(=O)N2CCCC2C(=O)NC(CC(C)C)C(=O)NCC(N)=O)NC(=O)C(CC(N)=O)NC(=O)C(CCC(N)=O)NC(=O)C(C(C)CC)NC1=O ; [2H][C@]1([C@@H](C)CC)NC(=O)C(Cc2ccc(OC)cc2)NC(=O)CCCSCC(C(=O)N2CCCC2C(=O)NC(CC(C)C)C(=O)NCC(N)=O)NC(=O)C(CC(N)=O)NC(=O)C(CCC(N)=O)NC1=O]

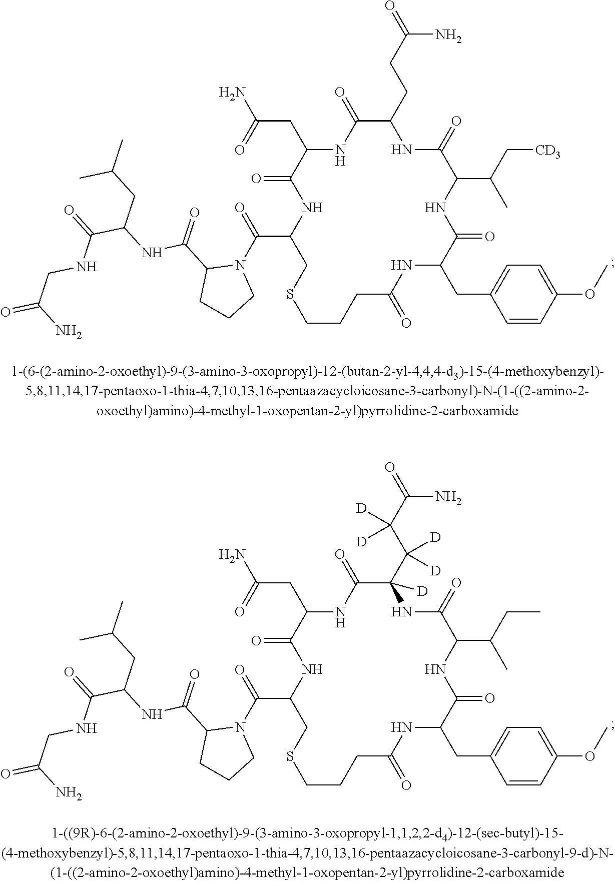 [SMILES: [2H]C([2H])([2H])CC(C)C1NC(=O)C(Cc2ccc(OC)cc2)NC(=O)CCCSCC(C(=O)N2CCCC2C(=O)NC(CC(C)C)C(=O)NCC(N)=O)NC(=O)C(CC(N)=O)NC(=O)C(CCC(N)=O)NC1=O ; [2H]C([2H])(C(N)=O)C([2H])([2H])[C@@]1([2H])NC(=O)C(C(C)CC)NC(=O)C(Cc2ccc(OC)cc2)NC(=O)CCCSCC(C(=O)N2CCCC2C(=O)NC(CC(C)C)C(=O)NCC(N)=O)NC(=O)C(CC(N)=O)NC1=O]

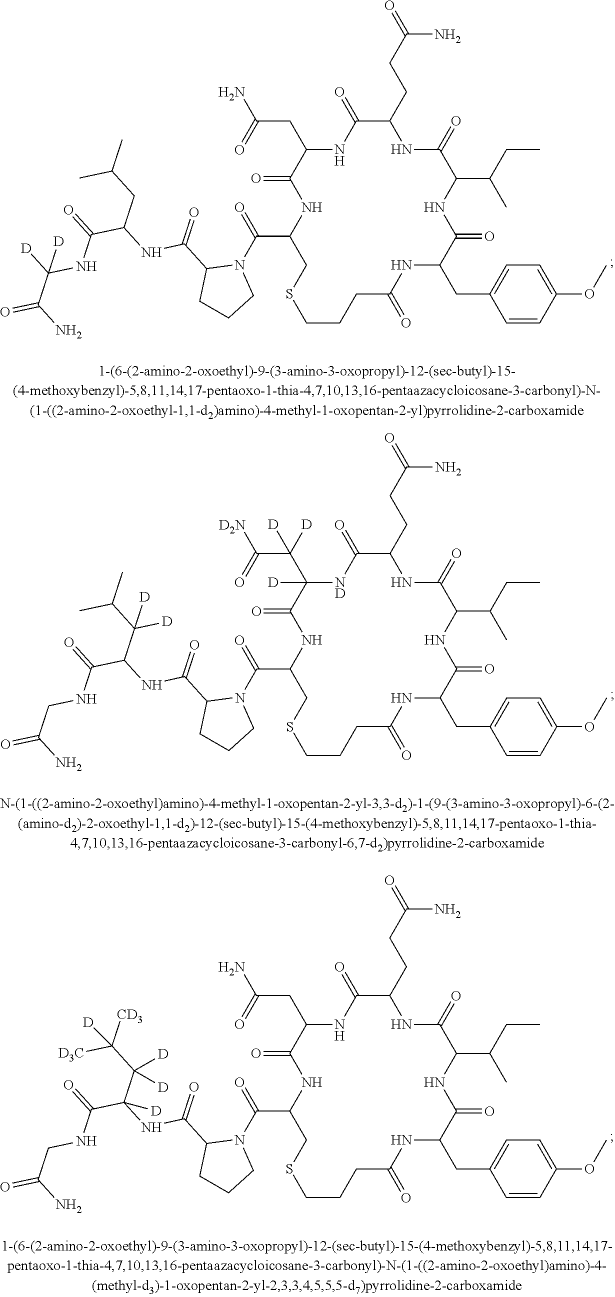

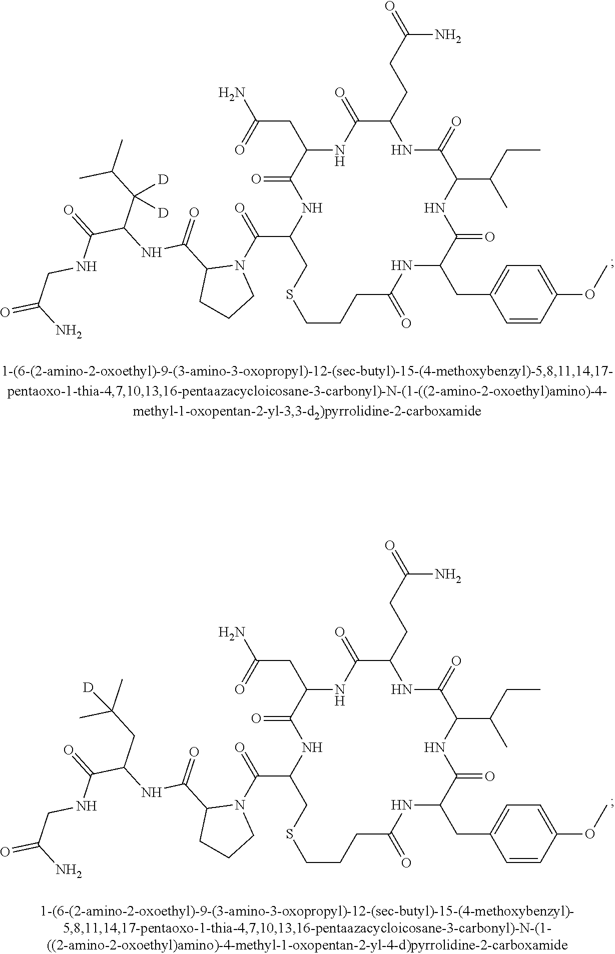 [SMILES: [2H]C([2H])(C(C)C)C(NC(=O)C1CCCN1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1)C(=O)NCC(N)=O ; [2H]C(C)(C)CC(NC(=O)C1CCCN1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1)C(=O)NCC(N)=O]

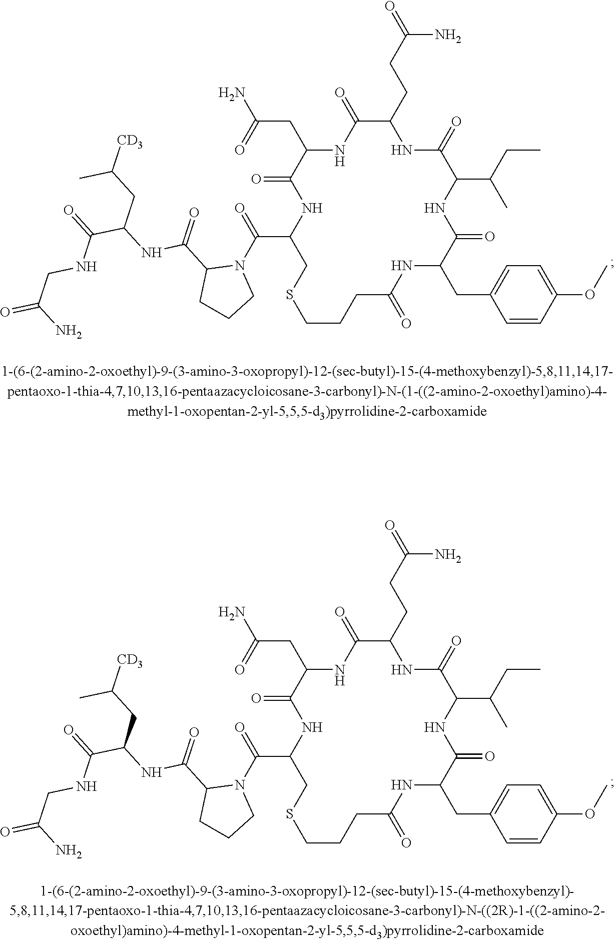 [SMILES: [2H]C([2H])([2H])C(C)CC(NC(=O)C1CCCN1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1)C(=O)NCC(N)=O ; [2H]C([2H])([2H])C(C)C[C@@H](NC(=O)C1CCCN1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1)C(=O)NCC(N)=O]

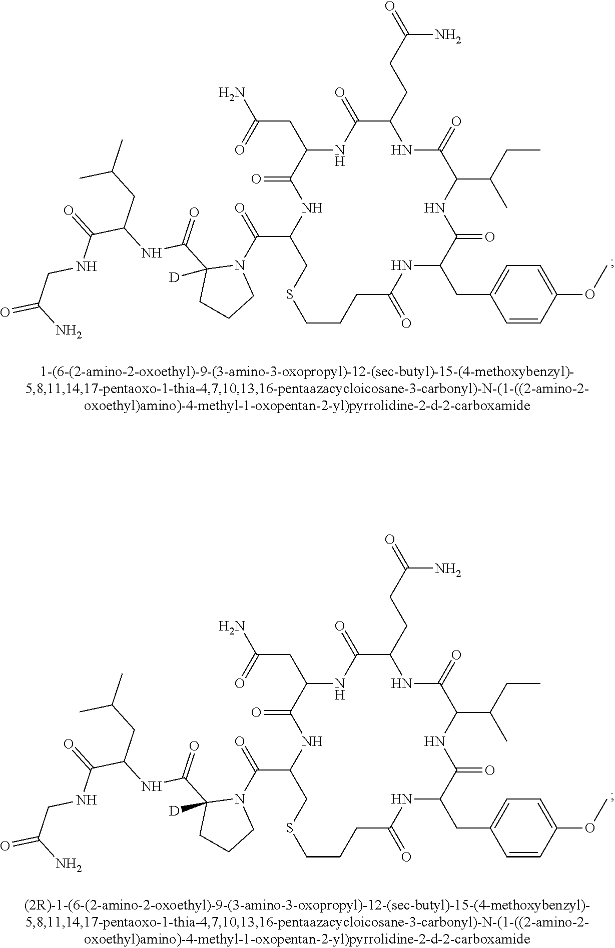 [SMILES: [2H]C1(C(=O)NC(CC(C)C)C(=O)NCC(N)=O)CCCN1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1 ; [2H][C@]1(C(=O)NC(CC(C)C)C(=O)NCC(N)=O)CCCN1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1]

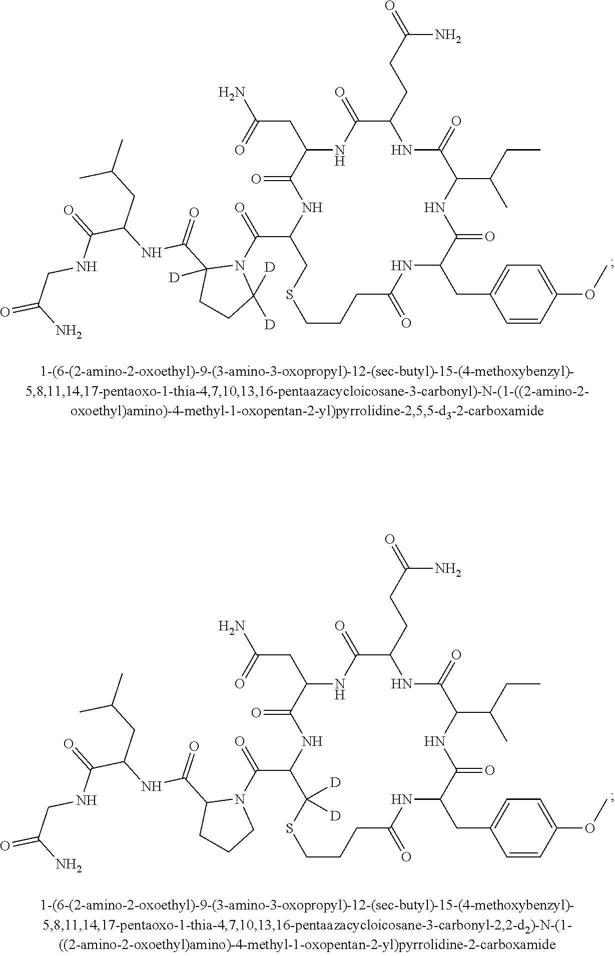 [SMILES: [2H]C1([2H])CCC([2H])(C(=O)NC(CC(C)C)C(=O)NCC(N)=O)N1C(=O)C1CSCCCC(=O)NC(Cc2ccc(OC)cc2)C(=O)NC(C(C)CC)C(=O)NC(CCC(N)=O)C(=O)NC(CC(N)=O)C(=O)N1 ; [2H]C1([2H])SCCCC(=O)NC(Cc2ccc(OC)cc2)C(=O)NC(C(C)CC)C(=O)NC(CCC(N)=O)C(=O)NC(CC(N)=O)C(=O)NC1C(=O)N1CCCC1C(=O)NC(CC(C)C)C(=O)NCC(N)=O]

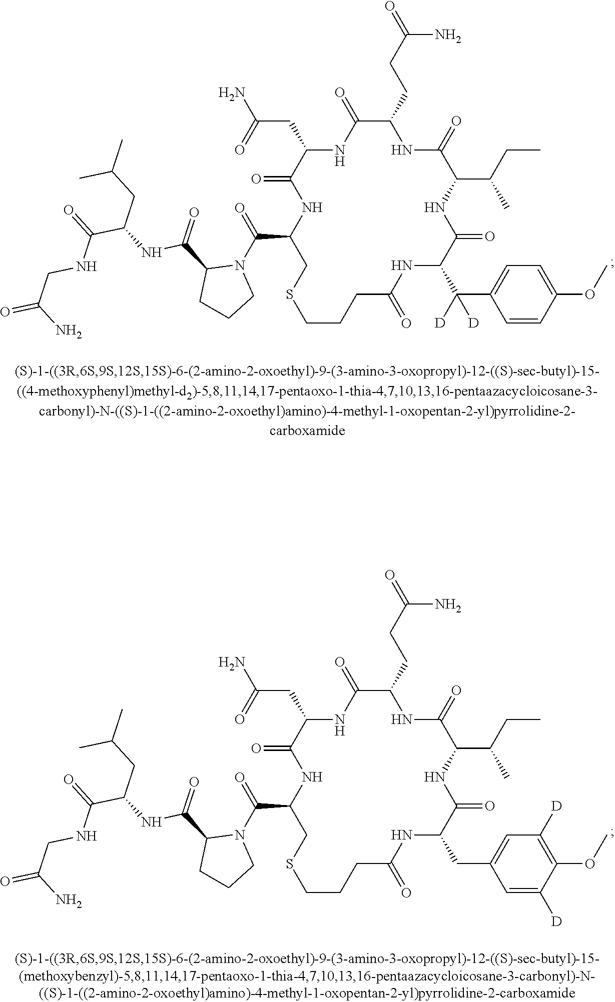 [SMILES: [2H]C([2H])(c1ccc(OC)cc1)[C@@H]1NC(=O)CCCSC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC(=O)[C@H]([C@@H](C)CC)NC1=O ; [2H]c1cc(C[C@@H]2NC(=O)CCCSC[C@@H](C(=O)N3CCC[C@H]3C(=O)N[C@@H](CC(C)C)C(=O)NCC(N)=O)NC(=O)[C@H](CC(N)=O)NC(=O)[C@H](CCC(N)=O)NC(=O)[C@H]([C@@H](C)CC)NC2=O)cc([2H])c1OC]

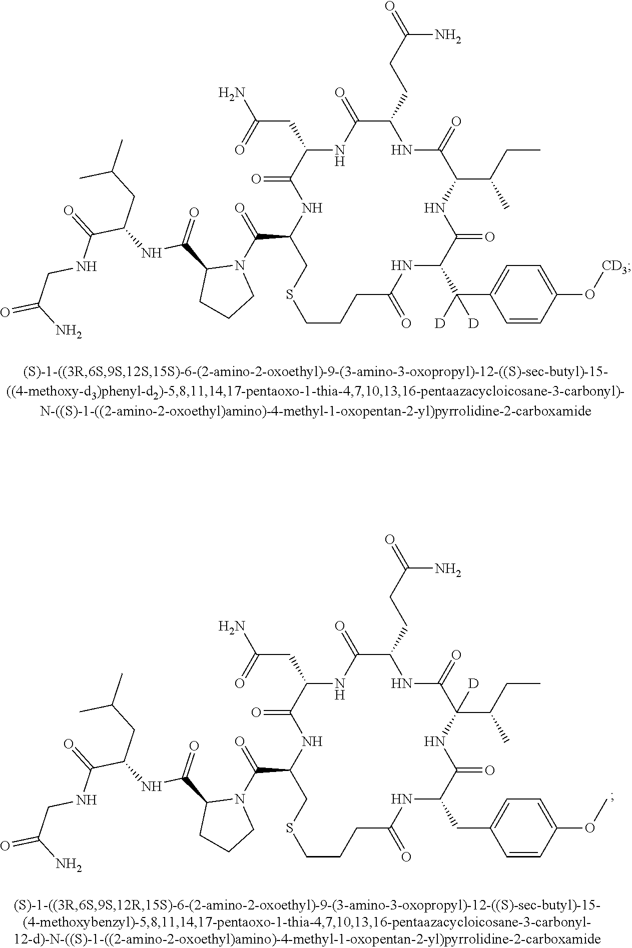 [SMILES: [2H]C([2H])([2H])Oc1ccc(C([2H])([2H])[C@@H]2NC(=O)CCCSC[C@@H](C(=O)N3CCC[C@H]3C(=O)N[C@@H](CC(C)C)C(=O)NCC(N)=O)NC(=O)[C@H](CC(N)=O)NC(=O)[C@H](CCC(N)=O)NC(=O)[C@H]([C@@H](C)CC)NC2=O)cc1 ; [2H][C@]1([C@@H](C)CC)NC(=O)[C@H](Cc2ccc(OC)cc2)NC(=O)CCCSC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

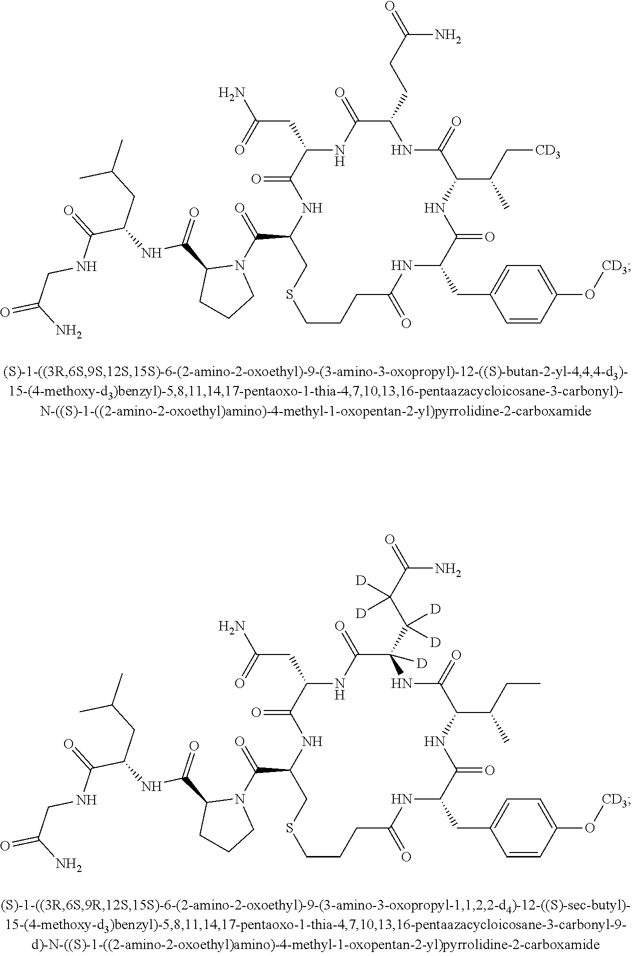 [SMILES: [2H]C([2H])([2H])C[C@H](C)[C@@H]1NC(=O)[C@H](Cc2ccc(OC([2H])([2H])[2H])cc2)NC(=O)CCCSC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O ; [2H]C([2H])([2H])Oc1ccc(C[C@@H]2NC(=O)CCCSC[C@@H](C(=O)N3CCC[C@H]3C(=O)N[C@@H](CC(C)C)C(=O)NCC(N)=O)NC(=O)[C@H](CC(N)=O)NC(=O)[C@@]([2H])(C([2H])([2H])C([2H])([2H])C(N)=O)NC(=O)[C@H]([C@@H](C)CC)NC2=O)cc1]

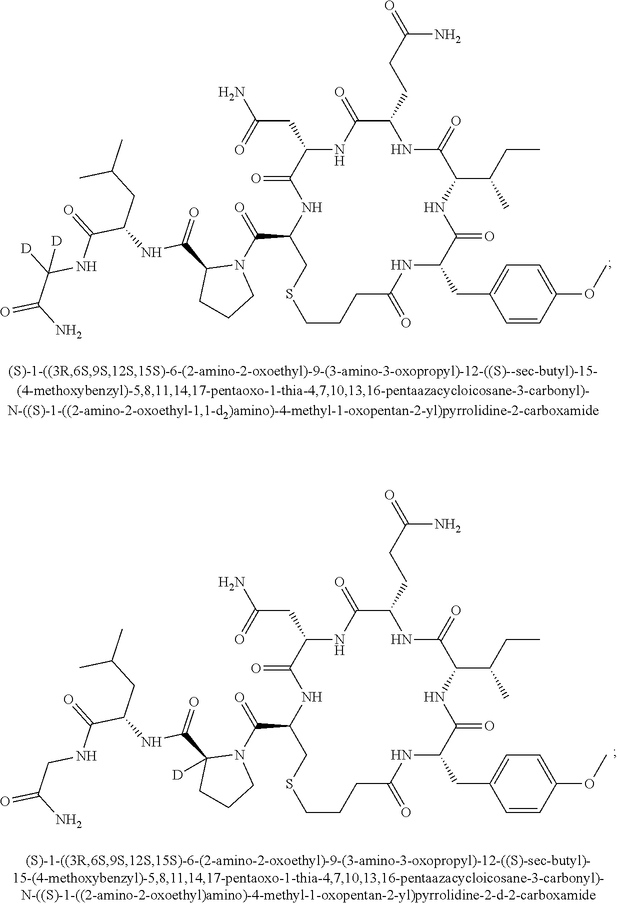 [SMILES: [2H]C([2H])(NC(=O)[C@H](CC(C)C)NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(N)=O ; [2H][C@@]1(C(=O)N[C@@H](CC(C)C)C(=O)NCC(N)=O)CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1]

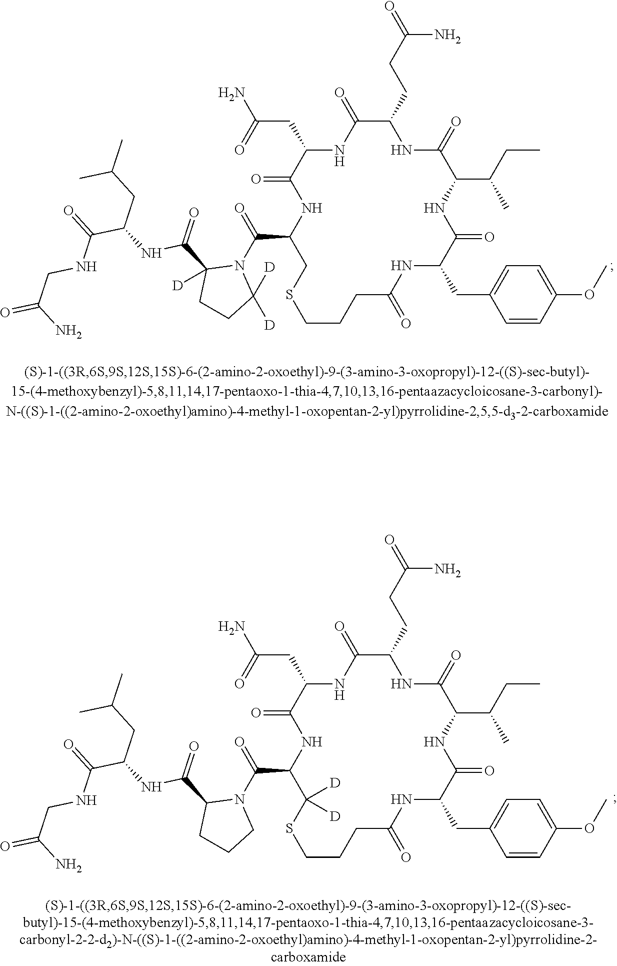 [SMILES: [2H]C1([2H])CC[C@@]([2H])(C(=O)N[C@@H](CC(C)C)C(=O)NCC(N)=O)N1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1 ; [2H]C1([2H])SCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N[C@@H]1C(=O)N1CCC[C@H]1C(=O)N[C@@H](CC(C)C)C(=O)NCC(N)=O]

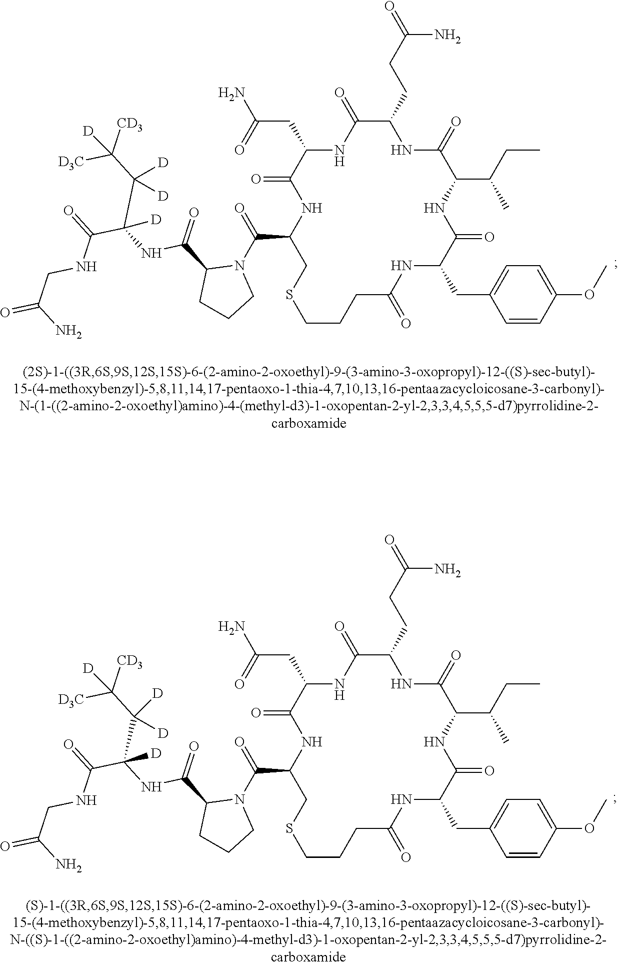 [SMILES: [2H]C([2H])([2H])C([2H])(C([2H])([2H])[2H])C([2H])([2H])[C@]([2H])(NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O ; [2H]C([2H])([2H])C([2H])(C([2H])([2H])[2H])C([2H])([2H])[C@]([2H])(NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

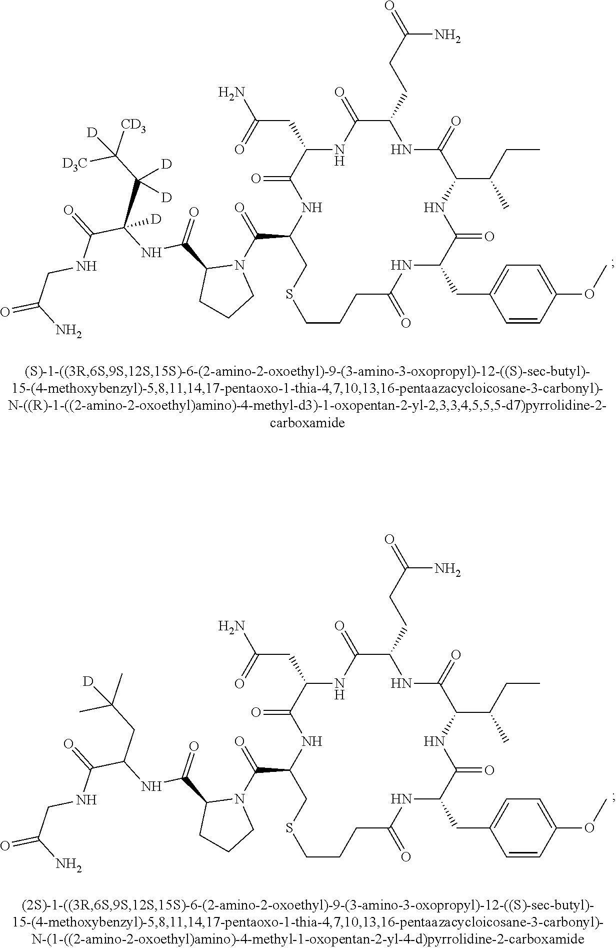 [SMILES: [2H]C([2H])([2H])C([2H])(C([2H])([2H])[2H])C([2H])([2H])[C@@]([2H])(NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O ; [2H]C(C)(C)CC(NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

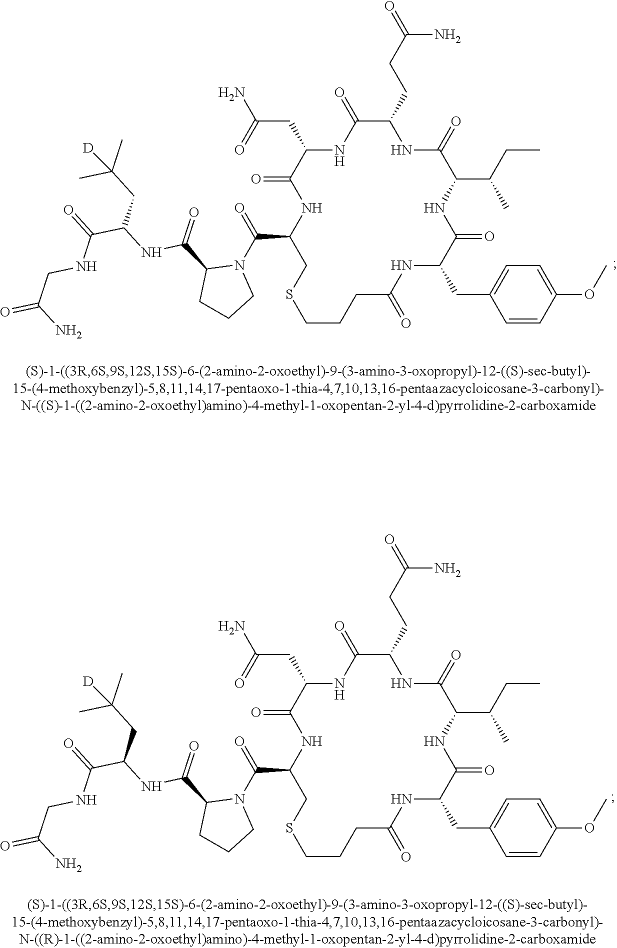

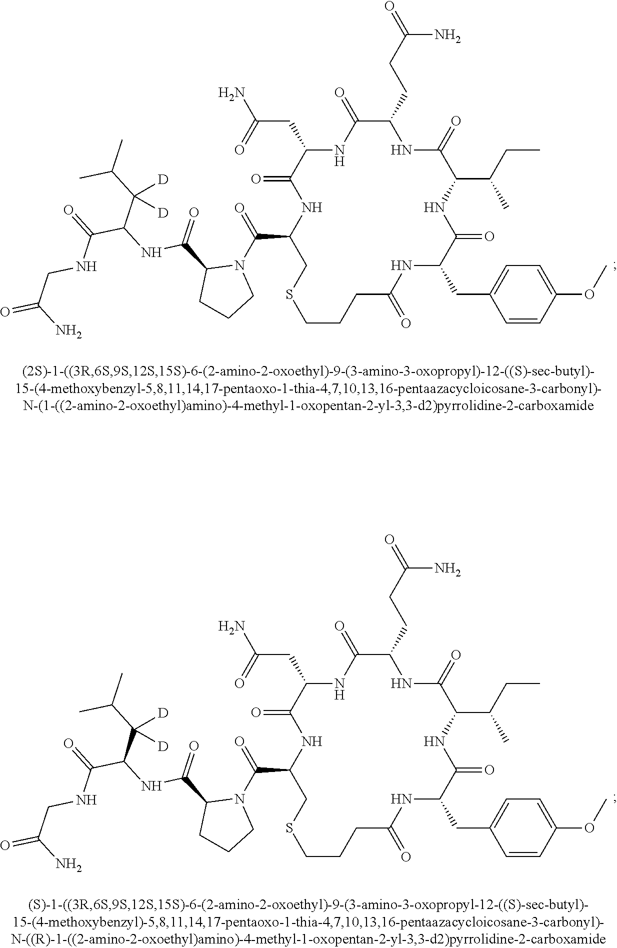 [SMILES: [2H]C([2H])(C(C)C)C(NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O ; [2H]C([2H])(C(C)C)[C@@H](NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

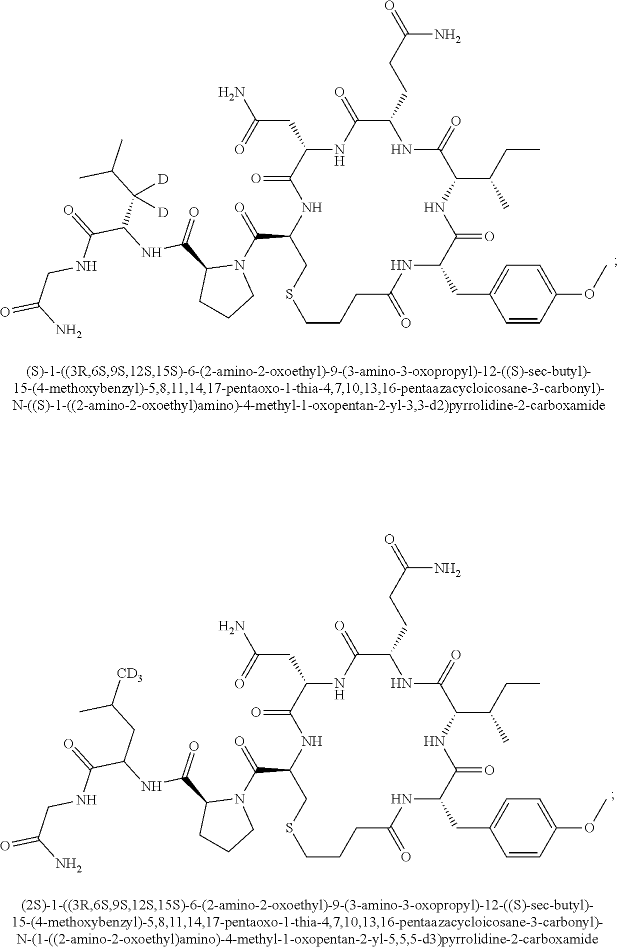

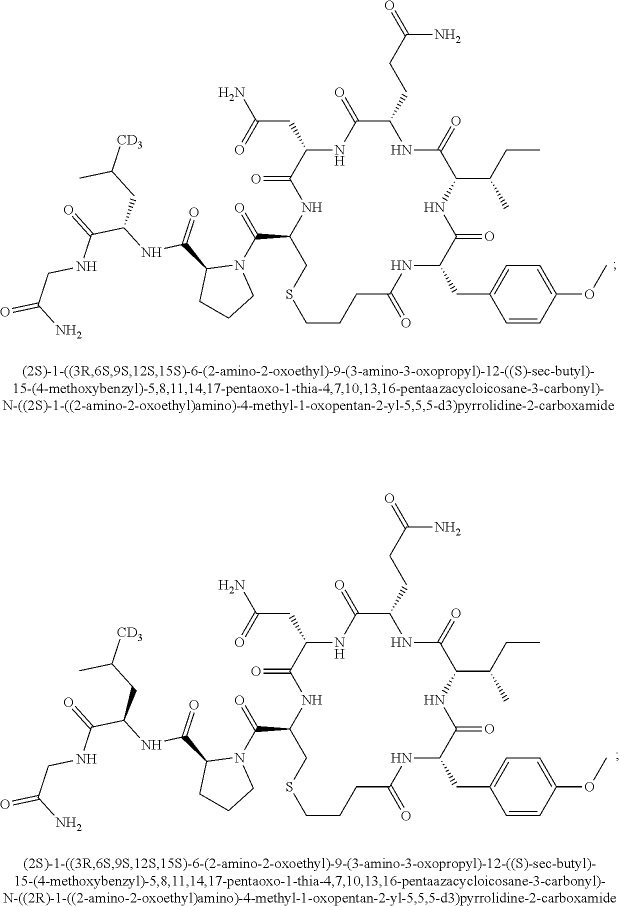 [SMILES: [2H]C([2H])([2H])C(C)C[C@H](NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O ; [2H]C([2H])([2H])C(C)C[C@@H](NC(=O)[C@@H]1CCCN1C(=O)[C@@H]1CSCCCC(=O)N[C@@H](Cc2ccc(OC)cc2)C(=O)N[C@@H]([C@@H](C)CC)C(=O)N[C@@H](CCC(N)=O)C(=O)N[C@@H](CC(N)=O)C(=O)N1)C(=O)NCC(N)=O]

or a pharmaceutically acceptable salt, stereoisomer, solvate, or hydrate thereof.

In at least one embodiment, the compound of Formula (A) is chosen from:

1-(6-(2-amino-2-oxoethyl)-9-(3-amino-3-oxopropyl)-12-(sec-butyl)-15-((4-methoxyphenyl)methyl-d2)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;

1-(6-(2-amino-2-oxoethyl)-9-(3-amino-3-oxopropyl)-12-(sec-butyl)-15-(4-(methoxy-d3)benzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;

1-((15S)-6-(2-amino-2-oxoethyl)-9-(3-amino-3-oxopropyl)-12-(sec-butyl)-15-((4-methoxyphenyl)methyl-d2)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;

1-((12R)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-12-d)-N-(1-((2-amino-2-oxoethyl)amino)-4-methyl-1-oxopentan-2-yl)pyrrolidine-2-carboxamide;

1-(6-(2-amino-2-oxoethyl)-9-(3-amino-3-oxopropyl)-12-(butan-2-yl-4,4,4-d3)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;

1-((9R)-6-(2-amino-2-oxoethyl)-9-(3-amino-3-oxopropyl-1,1,2,2-d4)-12-(sec-butyl)-15-(4-methoxybenzyl)-5,8,11,14,17-pentaoxo-1-thia-4,7,10,13,16-pentaazacycloicosane-3-carbonyl-9-d)-N-(1-((2-amino-2-oxoethyl)amino)-4-methyl-1-oxopentan-2-yl)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl-1,1-d2)amino)-4-methyl-1-oxopentan-2-yl)pyrrolidine-2-carboxamide;

N-(1-((2-amino-2-oxoethyl)amino)-4-methyl-1-oxopentan-2-yl-3,3-d2)-1-(9-(3-amino-3-oxopropyl)-6-(2-(amino-d2)-2-oxoethyl-1,1-d2)-12-(sec-butyl)-15-(4-methoxybenzyl)-5,8,11,14,17-pentaoxo-1-thia-4,7,10,13,16-pentaazacycloicosane-3-carbonyl-6,7-d2)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-(methyl-d3)-1-oxopentan-2-yl-2,3,3,4,5,5,5-d7)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-3,3-d2)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-4-d)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-5,5,5-d3)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-((2R)-1-((2-amino-2-oxoethyl)amino)-4-methyl-1-oxopentan-2-yl-5,5,5-d3)pyrrolidine-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-d-2-carboxamide;

(2R)-1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-d-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2,5,5-d3-2-carboxamide;

1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl-2,2-d2)-N-(1-((2-amino-2-oxoethyl)amino)-4-methyl-1-oxopentan-2-yl)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-((4-methoxyphenyl)methyl-d2)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-((4-(methoxy-d3)phenyl)methyl-d2)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;

(S)-1-((3R,6S,9S,12R,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-12-d)-N—((S)-1-((2-amino-2-oxoethyl)amino)-4-methyl-1-oxopentan-2-yl)pyrrolidine-2-carboxamide;

(S)-1-((3R,6S,9S,12S,15S)-6-(2-amino-2-oxoethyl)-9-(3-amino-3-oxopropyl)-12-((S)-butan-2-yl-4,4,4-d3)-15-(4-(methoxy-d3)benzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-d-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2,5,5-d3-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-2,2-d2)-N—((S)-1-((2-amino-2-oxoethyl)amino)-4-methyl-1-oxopentan-2-yl)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-(methyl-d3)-1-oxopentan-2-yl-2,3,3,4,5,5,5-d7)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-(methyl-d3)-1-oxopentan-2-yl-2,3,3,4,5,5,5-d7)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-(methyl-d3)-1-oxopentan-2-yl-2,3,3,4,5,5,5-d7)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-4-d)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl-4-d)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-methyl-1-oxopentan-2-yl-4-d)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-3,3-d2)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl-3,3-d2)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-methyl-1-oxopentan-2-yl-3,3-d2)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-5,5,5-d3)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((4R)-1-((2-amino-2-oxoethyl)amino)-4-methyl-1-oxopentan-2-yl-5,5,5-d3)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((4S)-1-((2-amino-2-oxoethyl)amino)-4-methyl-1-oxopentan-2-yl-5,5,5-d3)pyrrolidine-2-carboxamide;

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S)-1-((2-amino-2-oxoethyl)amino)-4-methyl-1-oxopentan-2-yl-5,5,5-d3)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S,4R)-1-((2-amino-2-oxoethyl)amino)-4-methyl-1-oxopentan-2-yl-5,5,5-d3)pyrrolidine-2-carboxamide;

(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S,4S)-1-((2-amino-2-oxoethyl)amino)-4-methyl-1-oxopentan-2-yl-5,5,5-d3)pyrrolidine-2-carboxamide; and

(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2R)-1-((2-amino-2-oxoethyl)amino)-4-methyl-1-oxopentan-2-yl-5,5,5-d3)pyrrolidine-2-carboxamide;

or a pharmaceutically acceptable salt, stereoisomer, solvate, or hydrate thereof.

### Methods of Preparation

In at least some embodiments, the present disclosure provides a method of making the deuterated compound of Formula (A) and Formula (Aa). In at least one embodiment, a deuterated compound of Formula (A) and Formula (Aa) is synthesized by solid phase peptide synthesis (SPPS) with a solid phase resin applying the Fmoc (9-fluorenylmethyloxycarbonyl) approach, in which the Fmoc group is used for temporary protection of α-amino groups. In some embodiments, the amino acid sequence is prepared in a stepwise manner by successive cycles of Fmoc deprotection before coupling of the following Fmoc-protected amino acid. In at least some embodiments, the amino acids chosen for SPPS may be any natural or unnatural amino acids. In some embodiments, the amino acids may be chosen from protected deuterated or undeuterated amino acids. In at least some embodiments, the amino acids may be chosen from N-α-Fluorenylmethoxycarbonyl-N-γ-trityl-L-asparagine (Fmoc-Asn(Trt)-OH), N-α-Fluorenylmethoxycarbonyl-terbutoxycarbonylpropyl-L-cysteine (Fmoc-Cys-((CH₂)₃—CO₂-tBu)-OH), N-α-Fluorenylmethoxycarbonyl-γ-trityl-L-glutamine (Fmoc-Gln(Trt)-OH), N-α-Fluorenylmethoxycarbonyl-glycine (Fmoc-Gly-OH), N-α-Fluorenylmethoxycarbonyl-L-isoleucine (Fmoc-Ile-OH), L-Leucine-d₁₀-N-Fmoc, DL-Leucine-d₁₀-N-Fmoc, N-α-Fluorenylmethoxycarbonyl-L-proline (Fmoc-Pro-OH), and N-α-Fluorenylmethoxycarbonyl-O-methyl-L-tyrosine (Fmoc-Tyr(Me)-OH).

In some embodiments, the amino acid sequence can be built up stepwise by successive cycles of Fmoc deprotection with a base in a solvent or solvent mixture before coupling of the following Fmoc-protected amino acid, up to the last desired amino acid.

In at least some embodiments, the linker used in Fmoc-synthesis may be, for example, a Wang linker. In at least some embodiments, the resin may be, for example, 4-methylbenzhydryl amine (MBHA) resin.

Fmoc deprotection may be accomplished by methods well known in the art.

In at least some embodiments, the linear protected peptide resin is then simultaneously cleaved from the resin and deprotected by treatment with an acid. In some embodiments the acid can be trifluoroacetic acid (TFA).

In some embodiments, a cyclized crude deuterated compound of Formula (A) or Formula (Aa) is obtained and may be kept in solution for direct application to preparative HPLC. In some embodiments, the crude deuterated compound of Formula (A) or Formula (Aa) is purified via one or more HPLC runs.

In at least one embodiment, the chromatographic purity of a deuterated compound of Formula (A) or Formula (Aa) is equal or greater than 85% as determined by HPLC. In one embodiment, the chromatographic purity of a deuterated compound of Formula (A) or Formula (Aa) is equal or greater than 88%. In at least one embodiment, the chromatographic purity of a deuterated compound of Formula (A) or Formula (Aa) is equal or greater than 90%. In at least one embodiment, the chromatographic purity of a deuterated compound of Formula (A) or Formula (Aa) is equal or greater than 91%. In at least one embodiment, the chromatographic purity of a deuterated compound of Formula (A) or Formula (Aa) is equal or greater than 92%. In at least one embodiment, the chromatographic purity of a deuterated compound of Formula (A) or Formula (Aa) is equal or greater than 93%. In at least one embodiment, the chromatographic purity of a deuterated compound of Formula (A) or Formula (Aa) is equal or greater than 94%. In at least one embodiment, the chromatographic purity of a deuterated compound of Formula (A) or Formula (Aa) is equal or greater than 95%. In at least one embodiment, the chromatographic purity of a deuterated compound of Formula (A) or Formula (Aa) is equal or greater than 96%. In at least one embodiment, the chromatographic purity of a deuterated compound of Formula (A) or Formula (Aa) is equal or greater than 97%. In at least one embodiment, the chromatographic purity of a deuterated compound of Formula (A) or Formula (Aa) is equal or greater than 98%. In at least one embodiment, the chromatographic purity of a deuterated compound of Formula (A) or Formula (Aa) is equal or greater than 99%. In at least one embodiment, a deuterated compound of Formula (A) or Formula (Aa) is not subject to chemical degradation, i.e., there is minimal or no change in chromatographic purity of a deuterated compound of Formula (A) or Formula (Aa) over a period of time. In addition, the pharmaceutical compositions of the present disclosure exhibit stability in that the concentration of a deuterated compound of Formula (A) or Formula (Aa) in solution does not change over time.

### Pharmaceutical Compositions

In at least some embodiments, the deuterated compounds of Formula (A) or Formula (Aa) may be used in a pharmaceutical composition. In some embodiments, the pharmaceutical composition may be administered intranasally daily for a period of time. In at least one embodiment, the pharmaceutical composition may be administered intranasally up to 3 times daily for chronic use. In at least one embodiment, the pharmaceutical composition may be administered in a volume of about 50 μL to about 200 μL into one nostril and then a volume of about 50 μL to about 200 μL into the second nostril, for a combined volume of about 100 μL to about 400 μL for both nostrils. In at least one embodiment, the pharmaceutical composition may be administered intranasally 3 times daily for 20 consecutive days. In at least one embodiment, the pharmaceutical composition may be administered in a volume of about 20 μL to about 200 μL into one nostril and then a volume of about 20 μL to about 200 μL into the second nostril, for a combined volume of about 40 μL to about 400 μL for both nostrils. In at least one embodiment, the pharmaceutical composition may be administered in a volume of about 25 μL to about 35 μL into one nostril and then a volume of about 25 μL to about 35 μL into the second nostril, for a combined volume of about 50 μL to about 70 μL for both nostrils. In one embodiment, the pharmaceutical composition may be administered in a volume of about 140 μL into one nostril and then a volume of about 140 μL into the second nostril, for a combined volume of about 280 μL for both nostrils.

### Methods of Treatment

In at least one embodiment, the disclosure provides a method of treating a subject suffering from, or susceptible to, a disease that is beneficially treated by a deuterated compound of Formula (A) comprising the step of administering to said subject an effective amount of the deuterated compound of Formula (A) disclosed herein.

In at least one embodiment, the improved deuterated compound of Formula (A) may be for use in (or in the manufacture of medicaments for) the treatment or prevention of neurodevelopmental disorders, including Prader-Willi syndrome, in a mammalian subject in need thereof. In at least one embodiment, a therapeutically-effective amount of a pharmaceutical composition of the present disclosure is administered to a subject suffering from Prader-Willi syndrome.

In some embodiments, the disclosure provides a method of administering an aqueous solution of a deuterated compound of Formula (A) intranasally comprising instructions for administration of the deuterated compound of Formula (A) or pharmaceutical composition over several days (e.g., 3-7 days). In at least one embodiment, a total daily dose of a deuterated compound of Formula (A) may be from about 1 mg/day to about 30 mg/day. In at least one embodiment, a total daily dose of the deuterated compound of Formula (A) may be from about 8.0 mg/day to about 30.0 mg/day. In at least one embodiment, a total daily dose of the deuterated compound of Formula (A) is from about 9.0 mg/day to about 29.0 mg/day. In one embodiment, a total daily dose of the deuterated compound of Formula (A) may be chosen from about 8.0 mg/day, about 9.0 mg/day, 10.0 mg/day, about 11.0 mg/day, about 12.0 mg/day, about 13.0 mg/day, about 14.0 mg/day, 15.0 mg/day, 16.0 mg/day, 17.0 mg/day, 18.0 mg/day, 19.0 mg/day, 20.0 mg/day, 21.0 mg/day, 22.0 mg/day, 23.0 mg/day, 24.0 mg/day, 25.0 mg/day, 26.0 mg/day, 27.0 mg/day, 28.0 mg/day, 29.0 mg/day, and about 30.0 mg/day. In another embodiment, a total daily dose of the deuterated compound of Formula (A) may be chosen from about 9.1 mg/day, about 9.2 mg/day, about 9.3 mg/day, about 9.4 mg/day, about 9.5 mg/day, about 9.6 mg/day, about 9.7 mg/day, about 9.8 mg/day, and about 9.9 mg/day. In at least one embodiment, a total daily dose of the deuterated compound of Formula (A) may be 9.6 mg/day. In one embodiment, a total daily dose of the deuterated compound of Formula (A) may be chosen from about 11.1 mg/day, about 11.2 mg/day, about 11.3 mg/day, about 11.4 mg/day, about 11.5 mg/day, about 11.6 mg/day, about 11.7 mg/day, about 11.8 mg/day, and about 11.9 mg/day. In at least one embodiment, a total daily dose of the deuterated compound of Formula (A) may be 11.4 mg/day. In one embodiment, a total daily dose of the deuterated compound of Formula (A) may be chosen from about 28.1 mg/day, about 28.2 mg/day, about 28.3 mg/day, about 28.4 mg/day, about 28.5 mg/day, about 28.6 mg/day, about 28.7 mg/day, about 28.8 mg/day, and about 28.9 mg/day. In at least one embodiment, a total daily dose of the deuterated compound of Formula (A) may be 28.8 mg/day. In at least one embodiment, the total daily dose may be divided into 3 equal doses. In some embodiments, the pharmaceutical compositions comprising a deuterated compound of Formula (A) may demonstrate reduced adverse side effects, decreased metabolic liability, enhanced subject compliance, and/or decreased population pharmacokinetic variability.

### EXAMPLES

The present disclosure may be better understood by reference to examples. The following example is intended for illustration purposes only and should not be construed as limiting the scope of the disclosure in any way. Further, the section headings used herein are for organizational purposes only and are not to be construed as limiting the subject matter described.

General Procedure for Solid-Phase Peptide Synthesis (SPPS)

The disclosed compounds are synthesized using SPPS and applying commercially available N-α-9-fluorenylmethyloxycarbonyl protected deuterated and/or undeuterated amino acids as building blocks in the assembling of the peptide from the C-terminal (Scheme 1 and Scheme 2). The C-terminal (e.g. —COOH group) of the first residue (e.g. glycyl residue; see steps 1 and 2 in Scheme 2) is coupled to, for example, a rink amide MBHA-resin via a rink amide linker after the 9-fluorenylmethyloxycarbonyl (Fmoc) group present in the rink amide MBHA-resin is first removed using a base (e.g. piperidine) (Scheme 2). Non-limiting examples of building blocks are both deuterated and undeuterated natural and unnatural amino acids. For example, as illustrated in Scheme 2, the amino acids for the SPPS may be chosen from protected deuterated or undeuterated amino acids, such as N-α-Fluorenylmethoxycarbonyl-N-γ-trityl-L-asparagine (Fmoc-Asn(Trt)-OH), N-α-Fluorenylmethoxycarbonyl-terbutoxycarbonylpropyl-L-cysteine (Fmoc-Cys-((CH₂)₃—CO₂-tBu)-OH), N-α-Fluorenylmethoxycarbonyl-γ-trityl-L-glutamine (Fmoc-Gln(Trt)-OH), N-α-Fluorenylmethoxycarbonyl-glycine (Fmoc-Gly-OH), N-α-Fluorenylmethoxycarbonyl-L-isoleucine (Fmoc-Ile-OH), L-Leucine-d₁₀-N-Fmoc, DL-Leucine-d₁₀-N-Fmoc, N-α-Fluorenylmethoxycarbonyl-L-proline (Fmoc-Pro-OH), and N-α-Fluorenylmethoxycarbonyl-O-methyl-L-tyrosine (Fmoc-Tyr(Me)-OH). Furthermore, non-limiting examples of coupling solvents are DMF, DCM, and DMF/DCM.

Other resins can be used. The amino acid sequence is built up stepwise by successive cycles of Fmoc deprotection with a base in a solvent or solvent mixture before coupling of the following Fmoc amino acid, up to the last desired amino acid (e.g. O-methyl tyrosine in Scheme 2).

Cleavage from Resin and Cyclization of Derivatives

The linear protected peptide resin is then simultaneously cleaved from the resin and deprotected by treatment with an acid, such as trifluoroacetic acid, and water as scavenger (see step 6 in Scheme 1; see step 1 in Scheme 3).

The cyclized crude deuterated compound of Formula (A) and Formula (Aa) may be kept in solution for direct application to preparative HPLC. The crude compound of Formula (A) and Formula (Aa) are applied to a preparative RPC (Reverse-phase chromatography) column. The main pool obtained after RPC may be applied to another preparative RPC column if a higher purity is desired.

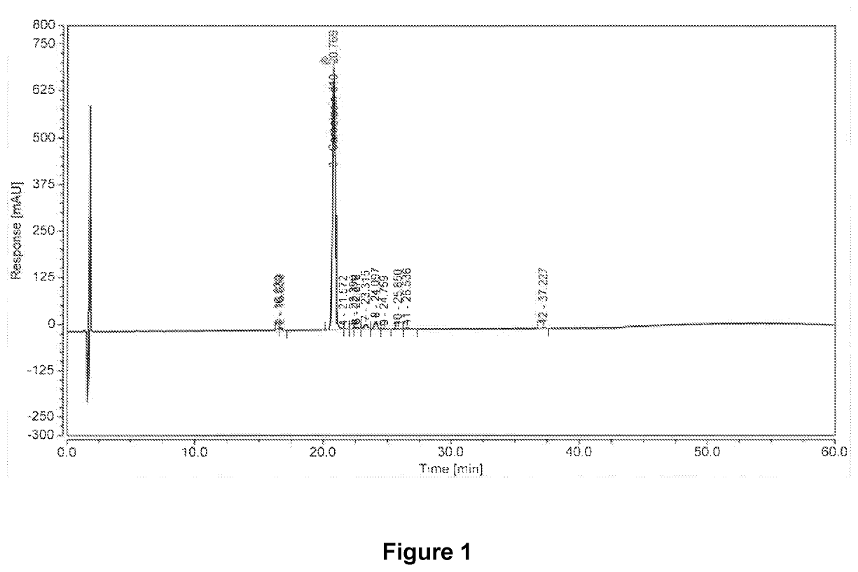

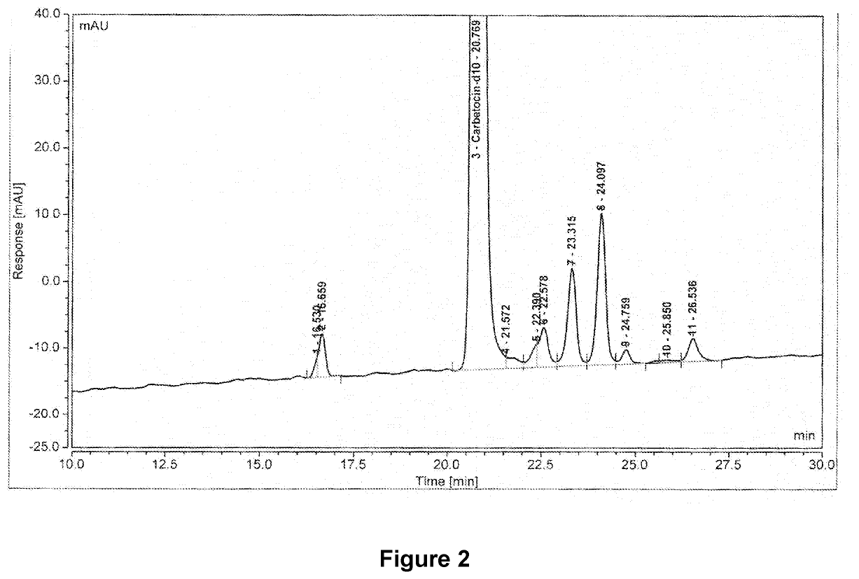

An illustrative compound of Formula (A) is compound (11), which is (S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-(methyl-d3)-1-oxopentan-2-yl-2,3,3,4,5,5,5-d7)pyrrolidine-2-carboxamide (i.e., [Leu-d₁₀]-Carbetocin or Carbetocin-d₁₀) shown in Scheme 3. Compound (11) was obtained as a white powder with a purity of ≥91% (see Table 1 below and FIGS. 1 and 2). As can be observed from FIGS. 1 and 2, Carbetocin-d₁₀ had a retention time of 20.769 minutes in an HPLC run and a relative area compared to the other peaks found in the chromatogram of 90.98% (see Table 1). Mass spectral analysis exhibited the correct molecular ion (997.8 mu).

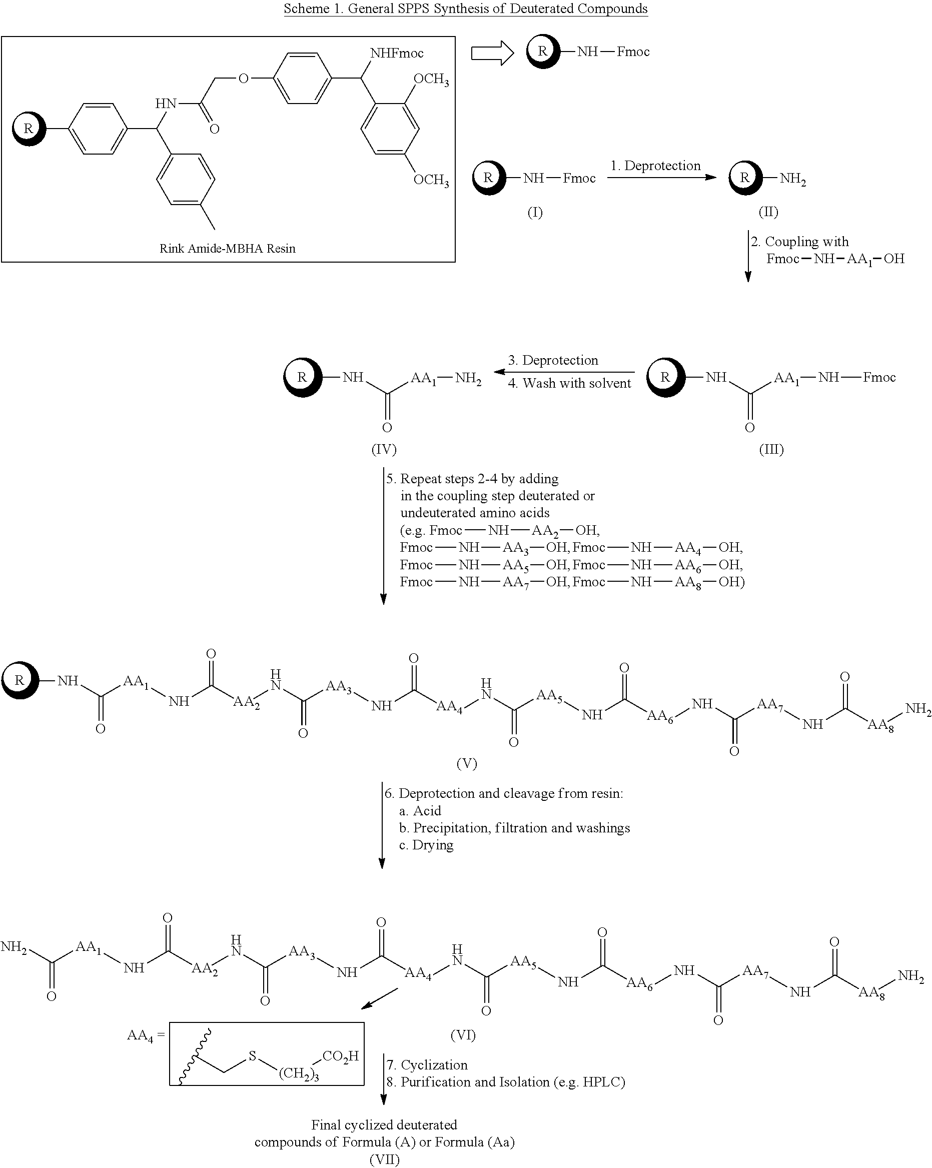

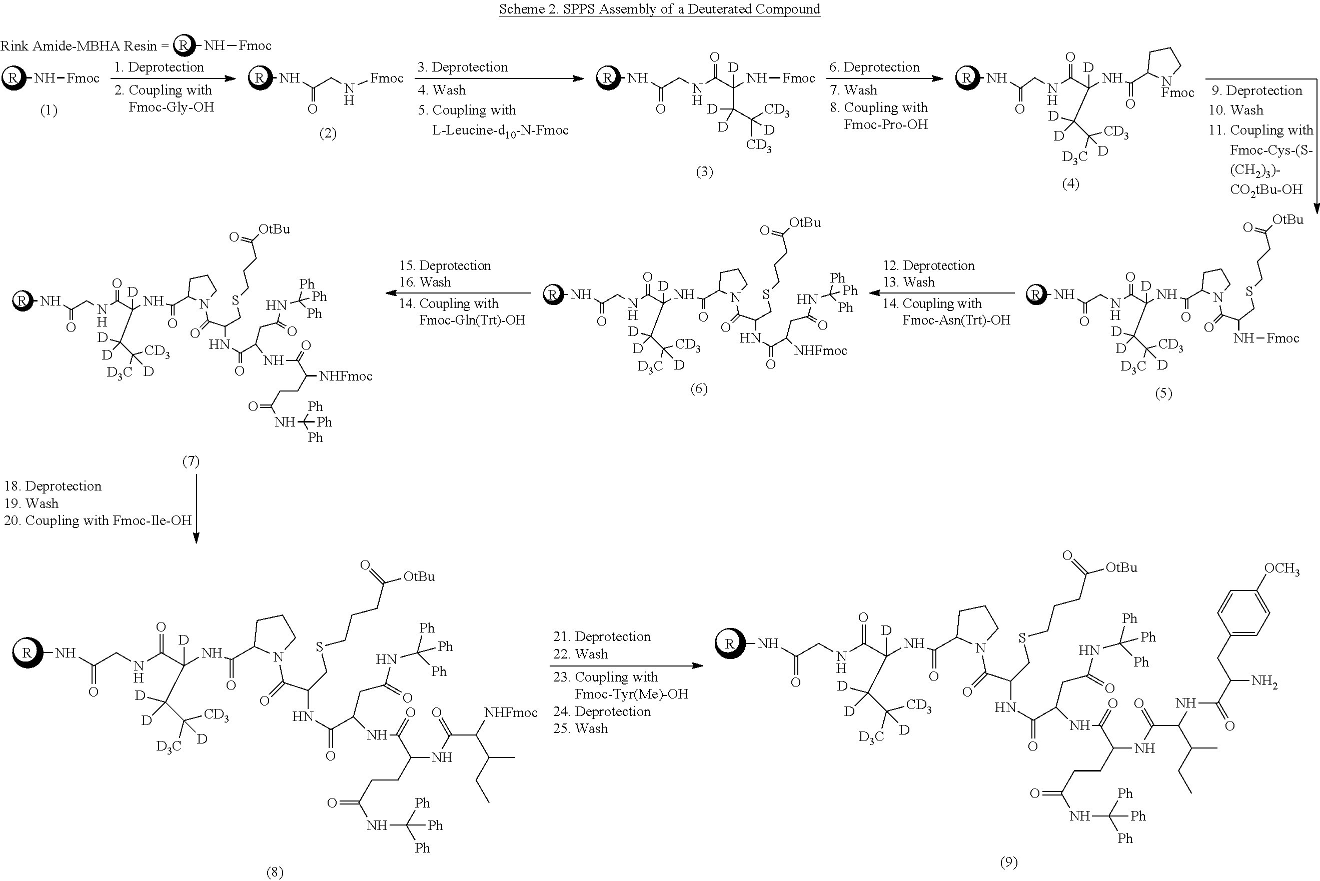

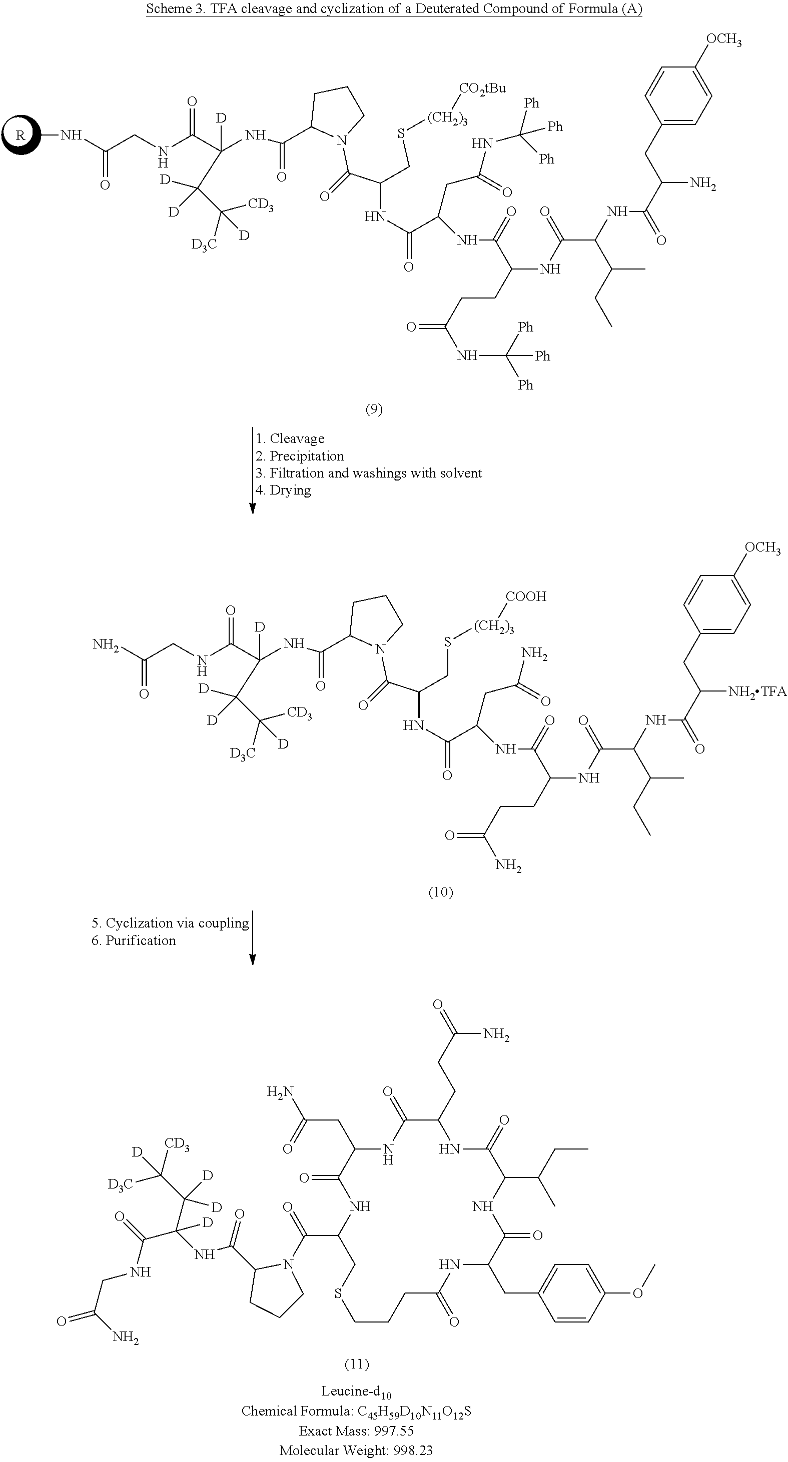

TABLE 1

| | | Retention | | | Relative | Relative | |
|---|---|---|---|---|---|---|---|
| | | Time | Area | Height | Area | Height | |
| No. | Peak Name | min | mAU * min | mAU | % | % | RRT |
| 1 | Carbetocin- | 16.530 | 0.257 | 3.013 | 0.14 | 0.39 | 0.796 |
| 2 | d10 | 16.659 | 1.290 | 6.535 | 0.69 | 0.86 | 0.802 |
| 3 | | 20.769 | 170.702 | 697.227 | 90.98 | 91.32 | 1.000 |
| 4 | | 21.572 | 0.615 | 1.751 | 0.33 | 0.23 | 1.039 |
| 5 | | 22.390 | 0.662 | 3.453 | 0.35 | 0.45 | 1.078 |
| 6 | | 22.578 | 1.721 | 5.952 | 0.92 | 0.78 | 1.087 |
| 7 | | 23.315 | 4.066 | 14.679 | 2.17 | 1.92 | 1.123 |
| 8 | | 24.097 | 5.976 | 22.687 | 3.19 | 2.97 | 1.160 |
| 9 | | 24.759 | 0.560 | 2.151 | 0.30 | 0.28 | 1.192 |
| 10 | | 25.850 | 0.178 | 0.383 | 0.10 | 0.05 | 1.245 |
| 11 | | 26.536 | 1.079 | 3.426 | 0.58 | 0.45 | 1.278 |
| 12 | | 37.227 | 0.520 | 2.210 | 0.28 | 0.29 | 1.792 |
| Total: | | | 187.628 | 763.465 | 100.00 | 100.00 | |

What is claimed is:

1. A deuterated compound of Formula (A), or a pharmaceutically acceptable salt, stereoisomer, solvate, or hydrate thereof: 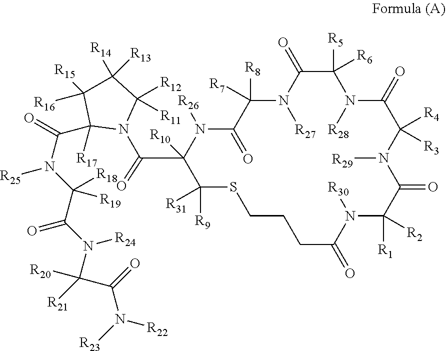
wherein:
R₁, R₃, R₅, R₇, R₉, R₁₀, R₁₁, R₁₂, R₁₃, R₁₄, R₁₅, R₁₆, R₁₇, R₁₈, R₂₀, R₂₁, R₂₂, R₂₃, R₂₄, R₂₅, R₂₆, R₂₇, R₂₈, R₂₉, R₃₀, and R₃₁ are independently chosen from H and D;
R₂ is chosen from 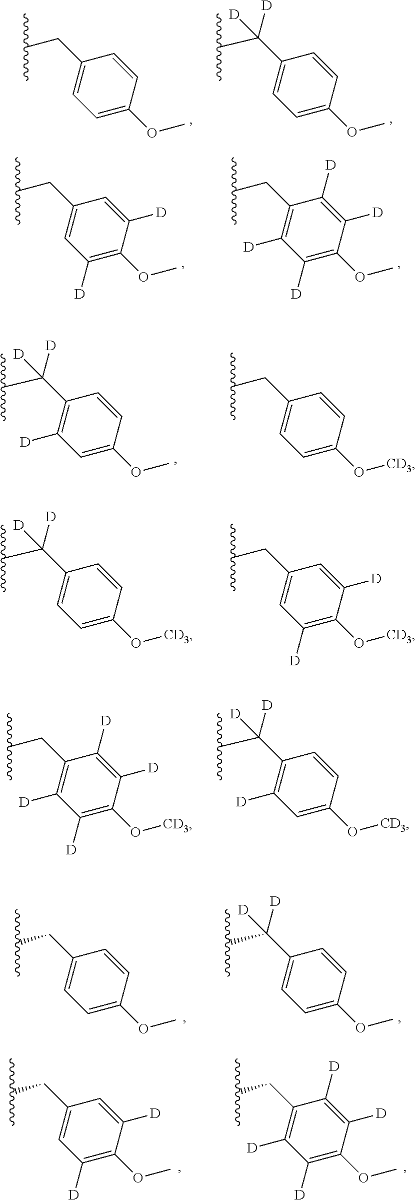 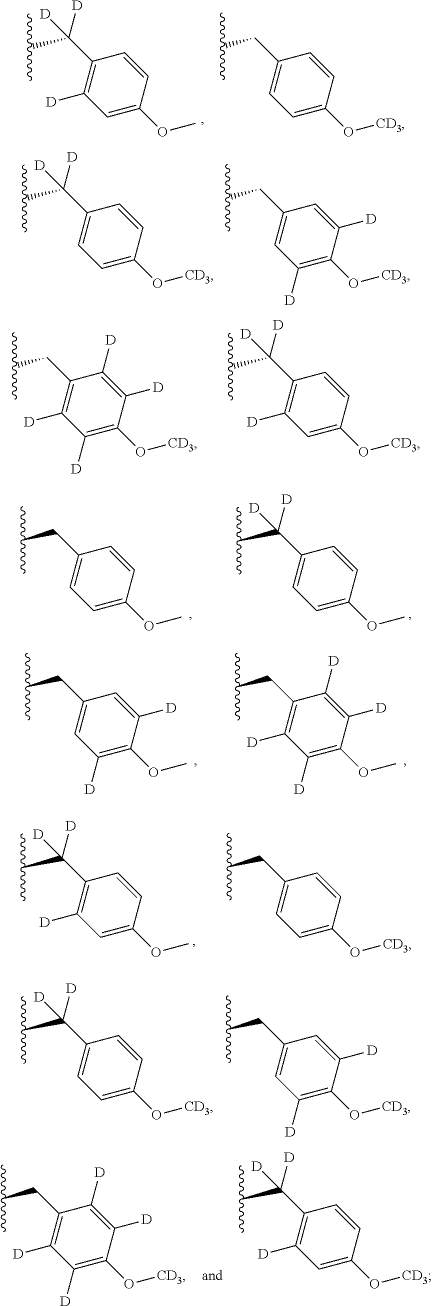
R₄ is chosen from 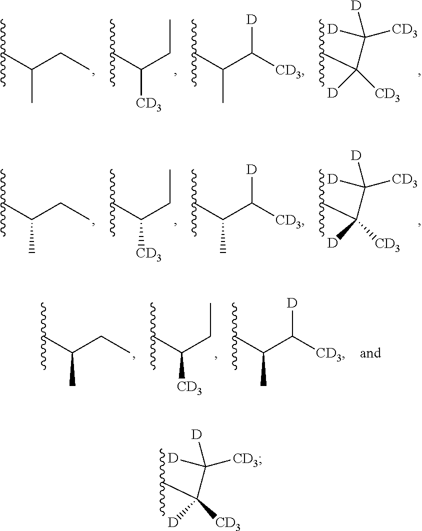
R₆ is chosen from 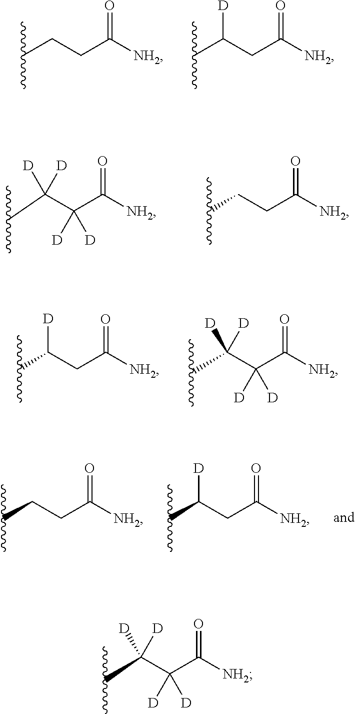
R₈ is chosen from 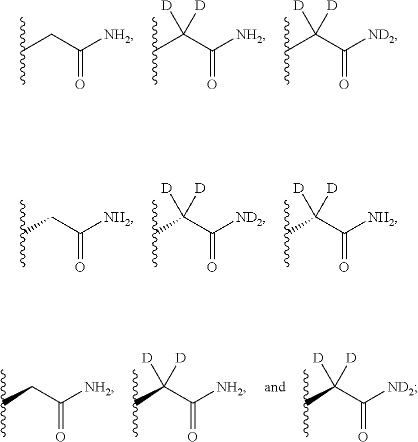
and
R₁₉ is chosen from

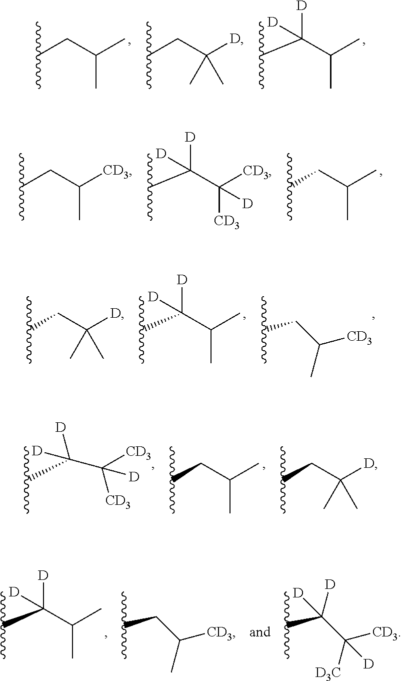

2. The deuterated compound according to claim 1, wherein:
R₁, R₃, R₅, R₇, R₉, R₁₀, R₁₁, R₁₂, R₁₃, R₁₄, R₁₅, R₁₆, R₁₇, R₂₀, R₂₁, R₂₂, R₂₃, R₂₄, R₂₅, R₂₆, R₂₇, R₂₈, R₂₉, R₃₀, and R₃₁ are independently H;
R₁₈ is chosen from H or D;
R₂ is chosen from 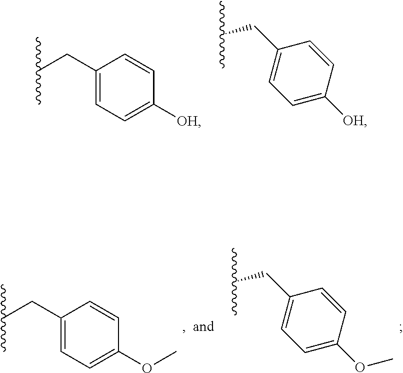
R₄ is chosen from 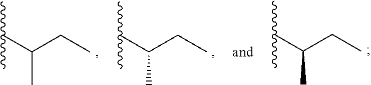
R₆ is chosen from 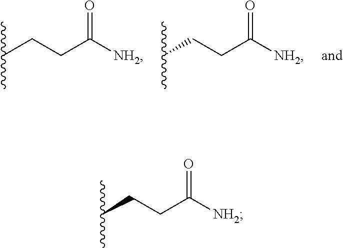
R₈ is chosen from 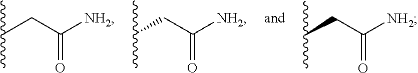
and
R₁₉ is chosen from

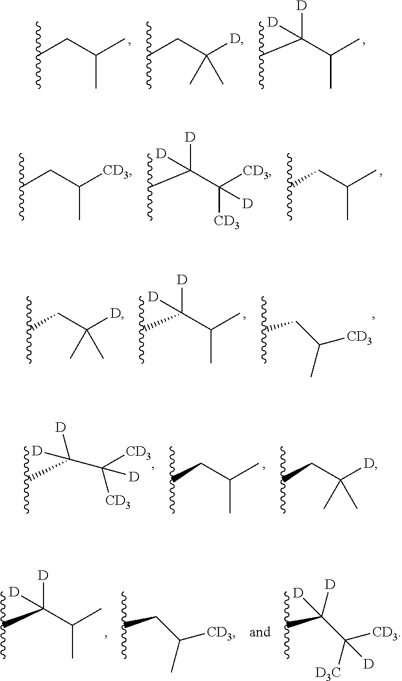

3. The deuterated compound according to claim 1, wherein:
R₁, R₃, R₅, R₇, R₉, R₁₀, R₁₁, R₁₂, R₁₃, R₁₄, R₁₅, R₁₆, R₁₇, R₂₀, R₂₁, R₂₂, R₂₃, R₂₄, R₂₅, R₂₆, R₂₇, R₂₈, R₂₉, R₃₀, and R₃₁ are independently H;
R₁₈ is D,
R₂ is 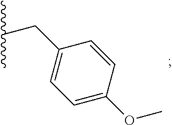 [SMILES: *Cc1ccc(OC)cc1]
R₄ is 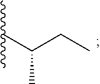 [SMILES: *[C@@H](C)CC ; ";"]
R₆ is 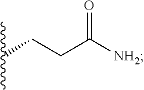
R₈ is 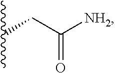
and
R₁₉ is

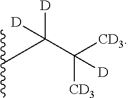 [SMILES: *C([2H])([2H])C([2H])(C([2H])([2H])[2H])C([2H])([2H])[2H]]

4. The deuterated compound according to claim 3, wherein the compound is 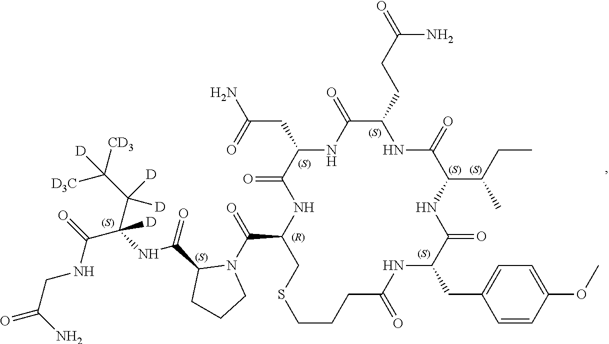
or a pharmaceutically acceptable salt, solvate, stereoisomer or hydrate thereof.

5. The deuterated compound according to claim 1, wherein the compound is chosen from:
1-(6-(2-amino-2-oxoethyl)-9-(3-amino-3-oxopropyl)-12-(sec-butyl)-15-((4-methoxyphenyl)methyl-d2)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-(6-(2-amino-2-oxoethyl)-9-(3-amino-3-oxopropyl)-12-(sec-butyl)-15-(4-(methoxy-d3)benzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-((15S)-6-(2-amino-2-oxoethyl)-9-(3-amino-3-oxopropyl)-12-(sec-butyl)-15-((4-methoxyphenyl)methyl-d2)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-((12R)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-12-d)-N-(1-((2-amino-2-oxoethyl)amino)-4-methyl-1-oxopentan-2-yl)pyrrolidine-2-carboxamide;
1-(6-(2-amino-2-oxoethyl)-9-(3-amino-3-oxopropyl)-12-(butan-2-yl-4,4,4-d3)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-((9R)-6-(2-amino-2-oxoethyl)-9-(3-amino-3-oxopropyl-1,1,2,2-d4)-12-(sec-butyl)-15-(4-methoxybenzyl)-5,8,11,14,17-pentaoxo-1-thia-4,7,10,13,16-pentaazacycloicosane-3-carbonyl-9-d)-N-(1-((2-amino-2-oxoethyl)amino)-4-methyl-1-oxopentan-2-yl)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl-1,1-d2)amino)-4-methyl-1-oxopentan-2-yl)pyrrolidine-2-carboxamide;
N-(1-((2-amino-2-oxoethyl)amino)-4-methyl-1-oxopentan-2-yl-3,3-d2)-1-(9-(3-amino-3-oxopropyl)-6-(2-(amino-d2)-2-oxoethyl-1,1-d2)-12-(sec-butyl)-15-(4-methoxybenzyl)-5,8,11,14,17-pentaoxo-1-thia-4,7,10,13,16-pentaazacycloicosane-3-carbonyl-6,7-d2)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-(methyl-d3)-1-oxopentan-2-yl-2,3,3,4,5,5,5-d7)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-3,3-d2)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-4-d)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-5,5,5-d3)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-((2R)-1-((2-amino-2-oxoethyl)amino)-4-methyl-1-oxopentan-2-yl-5,5,5-d3)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-d-2-carboxamide;
(2R)-1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-d-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2,5,5-d3-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl-2,2-d2)-N-(1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-((4-methoxyphenyl)methyl-d2)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-((4-(methoxy-d3)phenyl)methyl-d2)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;
(S)-1-((3R,6S,9S,12R,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-12-d)-N—((S)-1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-butan-2-yl-4,4,4-d3)-15-(4-(methoxy-d3)benzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl-1,1-d2)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-d-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2,5,5-d3-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-2,2-d2)-N—((S)-1-((2-amino-2-oxoethyl)amino)-4-methyl-1-oxopentan-2-yl)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-(methyl-d3)-1-oxopentan-2-yl-2,3,3,4,5,5,5-d7)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-(methyl-d3)-1-oxopentan-2-yl-2,3,3,4,5,5,5-d7)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-(methyl-d3)-1-oxopentan-2-yl-2,3,3,4,5,5,5-d7)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-4-d)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl-4-d)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-methyl-1-oxopentan-2-yl-4-d)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-3,3-d2)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl-3,3-d2)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-methyl-1-oxopentan-2-yl-3,3-d2)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-5,5,5-d3)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((4R)-1-((2-amino-2-oxoethyl)amino)-4-methyl-1-oxopentan-2-yl-5,5,5-d3)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((4S)-1-((2-amino-2-oxoethyl)amino)-4-methyl-1-oxopentan-2-yl-5,5,5-d3)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S)-1-((2-amino-2-oxoethyl)amino)-4-methyl-1-oxopentan-2-yl-5,5,5-d3)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S,4R)-1-((2-amino-2-oxoethyl)amino)-4-methyl-1-oxopentan-2-yl-5,5,5-d3)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S,4S)-1-((2-amino-2-oxoethyl)amino)-4-methyl-1-oxopentan-2-yl-5,5,5-d3)pyrrolidine-2-carboxamide; or
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2R)-1-((2-amino-2-oxoethyl)amino)-4-methyl-1-oxopentan-2-yl-5,5,5-d3)pyrrolidine-2-carboxamide;
or a pharmaceutically acceptable salt, stereoisomer, solvate, or hydrate thereof.

6. A pharmaceutical composition comprising the deuterated compound according to claim 1 and a pharmaceutically acceptable diluent or carrier.

7. A pharmaceutical composition comprising a deuterated compound and a pharmaceutically acceptable diluent or carrier, wherein the deuterated compound is
1-(6-(2-amino-2-oxoethyl)-9-(3-amino-3-oxopropyl)-12-(sec-butyl)-15-((4-methoxyphenyl)methyl-d2)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-(6-(2-amino-2-oxoethyl)-9-(3-amino-3-oxopropyl)-12-(sec-butyl)-15-(4-(methoxy-d3)benzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-((15S)-6-(2-amino-2-oxoethyl)-9-(3-amino-3-oxopropyl)-12-(sec-butyl)-15-((4-methoxyphenyl)methyl-d2)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-((12R)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-12-d)-N-(1-((2-amino-2-oxoethyl)amino)-4-methyl-1-oxopentan-2-yl)pyrrolidine-2-carboxamide;
1-(6-(2-amino-2-oxoethyl)-9-(3-amino-3-oxopropyl)-12-(butan-2-yl-4,4,4-d3)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-carboxamide;
1-((9R)-6-(2-amino-2-oxoethyl)-9-(3-amino-3-oxopropyl-1,1,2,2-d4)-12-(sec-butyl)-15-(4-methoxybenzyl)-5,8,11,14,17-pentaoxo-1-thia-4,7,10,13,16-pentaazacycloicosane-3-carbonyl-9-d)-N-(1-((2-amino-2-oxoethyl)amino)-4-methyl-1-oxopentan-2-yl)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl-1,1-d2)amino)-4-methyl-1-oxopentan-2-yl)pyrrolidine-2-carboxamide;
N-(1-((2-amino-2-oxoethyl)amino)-4-methyl-1-oxopentan-2-yl-3,3-d2)-1-(9-(3-amino-3-oxopropyl)-6-(2-(amino-d2)-2-oxoethyl-1,1-d2)-12-(sec-butyl)-15-(4-methoxybenzyl)-5,8,11,14,17-pentaoxo-1-thia-4,7,10,13,16-pentaazacycloicosane-3-carbonyl-6,7-d2)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-(methyl-d3)-1-oxopentan-2-yl-2,3,3,4,5,5,5-d7)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-3,3-d2)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-4-d)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-5,5,5-d3)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-((2R)-1-((2-amino-2-oxoethyl)amino)-4-methyl-1-oxopentan-2-yl-5,5,5-d3)pyrrolidine-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-d-2-carboxamide;
(2R)-1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2-d-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl)pyrrolidine-2,5,5-d3-2-carboxamide;
1-(6-(2-amino-2-oxoethyl)-9-(3-amino-3-oxopropyl)-12-(sec-butyl)-15-(4-methoxybenzyl)-5,8,11,14,17-pentaoxo-1-thia-4,7,10,13,16-pentaazacycloicosane-3-carbonyl-2,2-d2)-N-(1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-((4-methoxyphenyl)methyl-d2)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-((4-(methoxy-d3)phenyl)methyl-d2)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;
(S)-1-((3R,6S,9S,12R,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-12-d)-N—((S)-1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-butan-2-yl-4,4,4-d3)-15-(4-(methoxy-d3)benzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl-1,1-d2)amino)-4-methyl-1-oxopentan-2-yl)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2-d-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl)pyrrolidine-2,5,5-d3-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl-2,2-d2)-N—((S)-1-((2-amino-2-oxoethyl)amino)-4-methyl-1-oxopentan-2-yl)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-(methyl-d3)-1-oxopentan-2-yl-2,3,3,4,5,5,5-d7)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-(methyl-d3)-1-oxopentan-2-yl-2,3,3,4,5,5,5-d7)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-(methyl-d3)-1-oxopentan-2-yl-2,3,3,4,5,5,5-d7)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-4-d)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl-4-d)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-methyl-1-oxopentan-2-yl-4-d)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-3,3-d2)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-methyl-1-oxopentan-2-yl-3,3-d2)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((R)-1-((2-amino-2-oxoethyl)amino)-4-methyl-1-oxopentan-2-yl-3,3-d2)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-(1-((2-amino-2-oxoethyl)amino)-4-methyl-1-oxopentan-2-yl-5,5,5-d3)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((4R)-1-((2-amino-2-oxoethyl)amino)-4-methyl-1-oxopentan-2-yl-5,5,5-d3)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((4S)-1-((2-amino-2-oxoethyl)amino)-4-methyl-1-oxopentan-2-yl-5,5,5-d3)pyrrolidine-2-carboxamide;
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S)-1-((2-amino-2-oxoethyl)amino)-4-methyl-1-oxopentan-2-yl-5,5,5-d3)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S,4R)-1-((2-amino-2-oxoethyl)amino)-4-methyl-1-oxopentan-2-yl-5,5,5-d3)pyrrolidine-2-carboxamide;
(S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2S,4S)-1-((2-amino-2-oxoethyl)amino)-4-methyl-1-oxopentan-2-yl-5,5,5-d3)pyrrolidine-2-carboxamide; or
(2S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N-((2R)-1-((2-amino-2-oxoethyl)amino)-4-methyl-1-oxopentan-2-yl-5,5,5-d3)pyrrolidine-2-carboxamide;
or a pharmaceutically acceptable salt, solvate, stereoisomer, or hydrate thereof.

8. An intranasal drug product comprising the pharmaceutical composition of claim 6 and a device for intranasal delivery.

9. An intranasal drug product comprising the pharmaceutical composition of claim 7 and a device for intranasal delivery.

10. A method for preventing or treating a neurodevelopmental disorder in a subject in need thereof, comprising administering a therapeutically-effective amount of the deuterated compound of Formula (A) according to claim 1 to the subject.

11. The method of claim 10, wherein the neurodevelopmental disorder is Prader-Willi syndrome.

12. A method for preventing or treating a neurodevelopmental disorder in a subject in need thereof, comprising administering a therapeutically-effective amount of the pharmaceutical composition according to claim 6 to the subject.

13. The method of claim 12, wherein the neurodevelopmental disorder is Prader-Willi syndrome.

14. A method for preventing or treating a neurodevelopmental disorder in a subject in need thereof, comprising administering a therapeutically-effective amount of the pharmaceutical composition of claim 7 to the subject.

15. The method of claim 14, wherein the neurodevelopmental disorder is Prader-Willi syndrome.

16. A method for preventing or treating a neurodevelopmental disorder in a subject in need thereof, comprising administering a therapeutically-effective amount of (S)-1-((3R,6S,9S,12S,15S)-6-(2-amino-2-oxoethyl)-9-(3-amino-3-oxopropyl)-12-((S)-sec-butyl)-15-(4-methoxybenzyl)-5,8,11,14,17-pentaoxo-1-thia-4,7,10,13,16-pentaazacycloicosane-3-carbonyl)-N—((S)-1-((2-amino-2-oxoethyl)amino)-4-(methyl-d3)-1-oxopentan-2-yl-2,3,3,4,5,5,5-d7)pyrrolidine-2-carboxamide, or a pharmaceutically acceptable salt, solvate, stereoisomer or hydrate thereof, to the subject.

17. The method of claim 16, wherein the neurodevelopmental disorder is Prader-Willi syndrome.
